(19) United States
(12) Patent Application Publication
(10) Pub. No.: US 2003/0220386 A1
(43) Pub. Date: Nov. 27, 2003

(54) Delta 1-pyrrolines for use as pesticides

(76) Inventors: Andrew Plant, Winnersh (GB); Albrecht Marhold, Leverkusen (DE); Rolf Grosser, Leverkusen (DE); Christoph Erdelen, Leichlingen (DE); Andreas Turberg, Haan (DE); Olaf Hansen, Langenfeld (DE)

Correspondence Address: BAYER CROPSCIENCE LP, 100 BAYER ROAD, PITTSBURGH, PA, 15205, US

(21) Appl. No.: 10/380,728

(22) PCT Filed: Sep. 10, 2001
(86) PCT No.: PCT/EP01/10430

(30) Foreign Application Priority Data
Sep. 22, 2000 (DE) 100 47 119.6
Oct. 17, 2000 (DE) 100 51 395.6

(51) Int. Cl. A01N 43/36; C07D 207/12

(52) U.S. Cl. 514/408; 548/577; 548/543

(57) ABSTRACT

Novel Δ¹-pyrrolines of the formula (I)

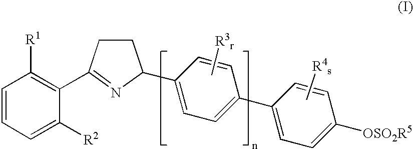

in which

R¹, R², R³, R⁴, R⁵, n, r and s are as defined in the description,

a plurality of processes for preparing these substances and their use for controlling pests, and novel intermediates.

The present invention relates to novel Δ¹-pyrrolines, to a plurality of processes for their preparation and to their use as pesticides.

It is already known that numerous Δ¹-pyrrolines have insecticidal properties (cf. WO 00/21958, WO 99/59968, WO 99/59967 and WO 98/22438). The activity of these substances is good; however, it is sometimes unsatisfactory.

This invention now provides Δ¹-pyrrolines of the formula (I)

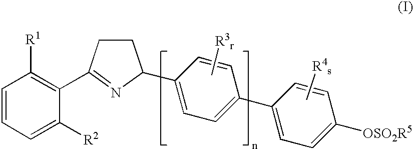

in which

n represents 0 or 1,

r and s independently of one another represent 0, 1 or 2,

R¹ represents halogen or methyl,

R² represents hydrogen or halogen,

R³ and R⁴ independently of one another represent halogen, alkyl, haloalkyl, alkoxy or haloalkoxy,

R⁵ represents alkyl, haloalkyl, phenyl which is in each case optionally mono- or polysubstituted by identical or different radicals from the list W¹ or represents —NR⁶R⁷,

W¹ represents halogen, alkyl, haloalkyl, alkoxy, haloalkoxy, alkylcarbonyl, alkoxycarbonyl or —S(O)_{q}R⁸,

R⁶ represents alkyl or haloalkyl,

R⁷ represents hydrogen, alkyl or haloalkyl,

R⁶ and R⁷ furthermore together represent alkylene or alkoxyalkylene,

R⁸ represents alkyl or haloalkyl and

q represents 0, 1 or 2.

Furthermore, it has been found that Δ¹-pyrrolines of the formula (I) are obtained when

A) aminoketones of the formula (II)

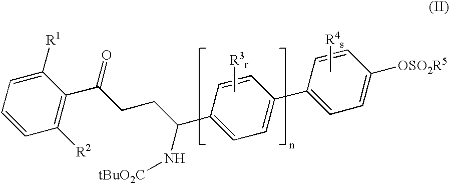

in which R¹, R², R³, R⁴, R⁵, n, r and s are as defined above

are treated with a Lewis acid or a protic acid, or

B) (bi)phenols of the formula (III)

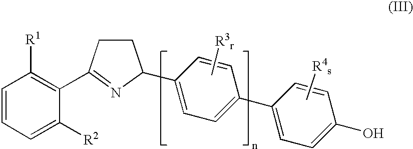

in which

R¹, R², R³, R⁴, n, r and s are as defined above

are reacted with a sulphonylating agent, if appropriate in the presence of an acid binder and if appropriate in the presence of a diluent, or

C) Δ¹-pyrrolines of the formula (I-a)

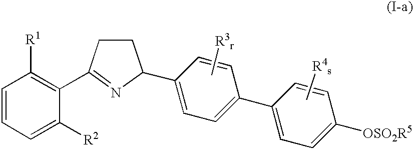

in which

R¹, R², R³, R⁴, R⁵, r and s are as defined above

are obtained by reacting pyrrolines of the formula (IV)

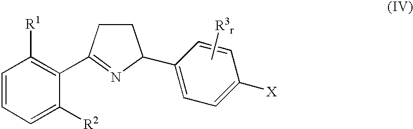

in which

R¹, R², R³ and r are as defined above and

X represents Br, Cl, I, —OSO₂CF₃ or —OSO₂(CF₂)₃CF₃,

initially with a diboronic ester in the presence of a catalyst, in the presence of an acid binder and, if appropriate, in the presence of a diluent and, if appropriate, after prior isolation of the resulting compounds of the formula (V)

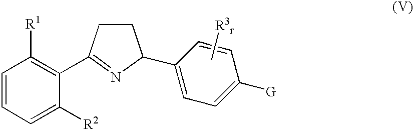

in which

R¹, R², R³ and r are as defined above and

G represents 4,4,5,5-tetramethyl-1,3,2-dioxaborolan-2-yl, 5,5-dimethyl-1,3,2-dioxaborinan-2-yl, 4,4,6-trimethyl-1,3,2-dioxaborinan-2-yl or 1,3,2-benzodioxaborol-2-yl,

reacting with iodides of the formula (VI)

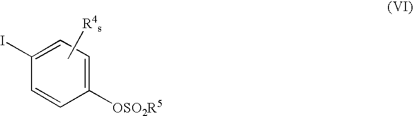

in which

R⁴, R⁵ and s are as defined above,

in the presence of a catalyst, in the presence of a diboronic ester, in the presence of an acid binder and, if appropriate, in the presence of a diluent.

Finally, it has been found that the compounds of the formula (I) according to the invention have very good insecticidal properties and can be used both in crop protection and in the protection of materials for controlling unwanted pests, such as insects.

Surprisingly, the insecticidal activity of the Δ¹-pyrrolines of the formula (I) according to the invention is considerably better than that of the constitutionally most similar prior-art Δ¹-pyrrolines of the same direction of action.

The formula (I) provides a general definition of the Δ¹-pyrrolines according to the invention.

n preferably represents 0 or 1.

r and s independently of one another preferably represent 0, 1 or 2.

R¹ preferably represents fluorine, chlorine, bromine or methyl.

R² preferably represents hydrogen, fluorine, chlorine or bromine.

R³ and R⁴ independently of one another preferably represent fluorine, chlorine, bromine, C₁-C₆-alkyl, C₁-C₆-haloalkyl, C₁-C₆-alkoxy or C₁-C₆-haloalkoxy.

R⁵ preferably represents C₁-C₆-alkyl, C₁-C₆-haloalkyl, phenyl which is in each case optionally mono- to tetrasubstituted by identical or different radicals from the list W¹ or represents —NR⁶R⁷.

W¹ preferably represents fluorine, chlorine, bromine, C₁-C₆-alkyl, C₁-C₆-haloalkyl, C₁-C₆-alkoxy, C₁-C₆-haloalkoxy, C₁-C₆-alkylcarbonyl, C₁-C₆-alkoxycarbonyl or —S(O)_{q}R⁸.

R⁶ preferably represents C₁-C₆-alkyl or C₁-C₆-haloalkyl.

R⁷ preferably represents hydrogen, C₁-C₆-alkyl or C₁-C₆-haloalkyl.

R⁶ and R⁷ furthermore together preferably represent C₃-C₆-alkylene or C₁-C₄-alkoxy-C₁-C₄-alkylene.

R⁸ preferably represents C₁-C₆-alkyl or C₁-C₆-haloalkyl.

q preferably represents 0, 1 or 2.

n particularly preferably represents 0 or 1.

r and s independently of one another particularly preferably represent 0, 1 or 2.

R¹ particularly preferably represents fluorine, chlorine or methyl.

R² particularly preferably represents hydrogen, fluorine or chlorine.

R³ and R⁴ independently of one another particularly preferably represent fluorine, chlorine, C₁-C₄-alkyl, C₁-C₄-haloalkyl, C₁-C₄-alkoxy, C₁-C₄-haloalkoxy.

R⁵ particularly preferably represents C₁-C₄-alkyl, C₁-C₄-haloalkyl, phenyl which is in each case optionally mono- to trisubstituted by identical or different radicals from the list W¹ or represents —NR⁶R⁷.

W¹ particularly preferably represents fluorine, chlorine, C₁-C₄-alkyl, C₁-C₄-haloalkyl, C₁-C₄-alkoxy, C₁-C₄-haloalkoxy, C₁-C₄-alkylcarbonyl, C₁-C₄-alkoxycarbonyl or —S(O)_{q}R⁸.

R⁶ particularly preferably represents C₁-C₄-alkyl or C₁-C₄-haloalkyl.

R⁷ particularly preferably represents hydrogen, C₁-C₄-alkyl or C₁-C₄-haloalkyl.

R⁶ and R⁷ furthermore together particularly preferably represent C₄-C₅-alkylene or —(CH₂)₂—O—(CH₂)₂—.

R⁸ particularly preferably represents C₁-C₄-alkyl or C₁-C₄-haloalkyl.

q particularly preferably represents 0, 1 or 2.

n very particularly preferably represents 0 or 1.

r and s independently of one another very particularly preferably represent 0 or 1.

R¹ very particularly preferably represent fluorine or chlorine.

R² very particularly preferably represent hydrogen or fluorine.

R³ and R⁴ independently of one another very particularly preferably represent fluorine, chlorine, methyl, ethyl, n-propyl, i-propyl, n-butyl, i-butyl, s-butyl, t-butyl, trifluoromethyl, trifluoroethyl, methoxy, ethoxy, n-propoxy, i-propoxy, n-butoxy, i-butoxy, s-butoxy, t-butoxy, trifluoromethoxy or trifluoroethoxy.

R⁵ very particularly preferably represents methyl, ethyl, n-propyl, i-propyl, n-butyl, i-butyl, s-butyl, t-butyl, trifluoromethyl, trifluoroethyl, nonafluorobutyl, phenyl which is in each case optionally mono- or disubstituted by identical or different radicals from the list W¹ or represents —NR⁶R⁷.

W¹ very particularly preferably represents fluorine, chlorine, methyl, ethyl, n-propyl, i-propyl, n-butyl, i-butyl, s-butyl, t-butyl, trifluoromethyl, trifluoroethyl, methoxy, ethoxy, n-propoxy, i-propoxy, n-butoxy, i-butoxy, s-butoxy, t-butoxy, trifluoromethoxy, trifluoroethoxy, —COCH₃, —CO₂CH₃, —SCF₃, —SCHF₂, —SOCF₃, —SOCHF₂, —SO₂CF₃ or —SO₂CHF₂.

R⁶ very particularly preferably represents methyl, ethyl, n-propyl, i-propyl, n-butyl, i-butyl, s-butyl, t-butyl, trifluoromethyl or trifluoroethyl.

R⁷ very particularly preferably represents hydrogen, methyl, ethyl, n-propyl, i-propyl, n-butyl, i-butyl, s-butyl, t-butyl, trifluoromethyl or trifluoroethyl.

Preference is furthermore given to compounds of the formula (I-a)

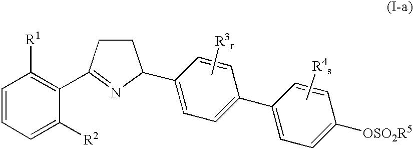

in which

R¹, R², R³, R⁴, R⁵, r and s are as defined above.

Preference is furthermore given to compounds of the formula (I-b)

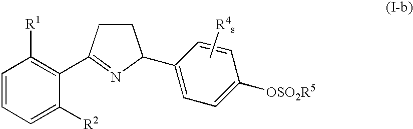

in which

R¹, R², R⁴, R⁵ and s are as defined above.

Moreover, preference is given to compounds of the formulae (I-a) and (I-b) in which R¹, R², R³, R⁴, R⁵, r and s each have the preferred meanings given above.

Moreover, preference is given to compounds of the formulae (I-a) and (I-b) in which R¹, R², R³, R⁴, R⁵, r and s each have the particularly preferred meanings given above.

Moreover, preference is given to compounds of the formulae (I-a) and (I-b) in which R¹, R², R³, R⁴, R⁵, r and s each have the very particularly preferred meanings given above.

Furthermore, very particular preference is given to (R)-configured compounds of the formula (I-c)

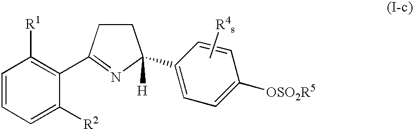

in which

R¹, R², R⁴, R⁵ and s are as defined above.

Furthermore, very particular preference is given to (R)-configured compounds of the formula (I-d)

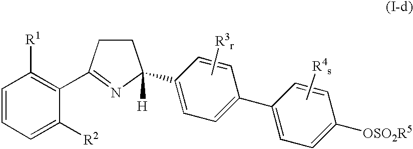

in which

R¹, R², R³, R⁴, R⁵, r and s are as defined above.

Enantiomerically pure compounds of the formulae (I-c) and (I-d) are obtained by customary procedures for resolving racemates, such as, for example by chromatography of the corresponding racemates on a chiral stationary phase. In this manner, it is possible to separate both racemic end products and racemic intermediates into the two enantiomers.

Saturated hydrocarbon radicals, such as alkyl, can in each case be straight-chain or branched as far as this is possible, including in combination with heteroatoms, such as, for example, in alkoxy.

Optionally substituted radicals can be mono- or polysubstituted, where in the case of polysubstitution the substituents can be identical or different. A plurality of radicals having the same indices, such as, for example, s radicals R⁴ for s=2, can be identical or different.

However, the abovementioned general or preferred radical definitions or illustrations can also be combined with one another as desired, i.e. including combinations between the respective ranges and preferred ranges. They apply both to the end products and, correspondingly, to precursors and intermediates.

Using 4′-[1-[(tert-butoxycarbonyl)amino]-4-(2,6-difluorophenyl)-4-oxobutyl]-1,1′-biphenyl-4-yl trifluoromethanesulphonate and trifluoroacetic acid (TFA) as starting materials, the course of the process (A) according to the invention can be illustrated by the formula scheme below.

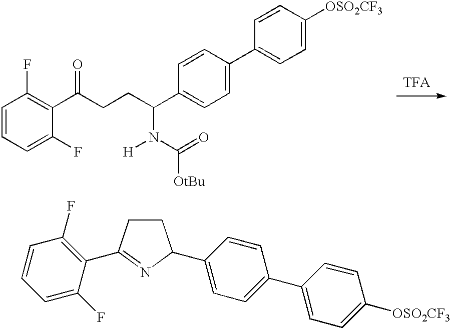

Using 4′-[5-(2,6-difluorophenyl)-3,4-dihydro-2H-pyrrol-2-yl]-1,1′-biphenyl-4-ol and trifluoromethanesulphonyl chloride (F₃CSO₂Cl) as starting materials, the course of the process (B) according to the invention can be illustrated by the formula scheme below.

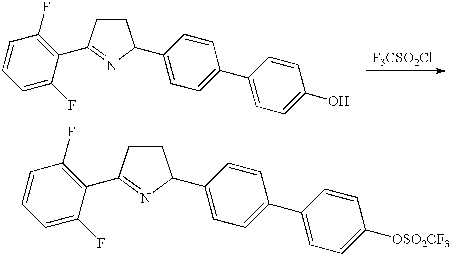

Using 5-(2,6-difluorophenyl)-2-(4-bromophenyl)-3,4-dihydro-2H-pyrrole, 4-iodophenyl trifluoromethanesulphonate, a palladium catalyst and 4,4,4′,4′,5,5,5′,5′-octamethyl-2,2′-bis-1,3,2-dioxaborolane as starting materials, the process (C) according to the invention can be illustrated by the formula scheme below.

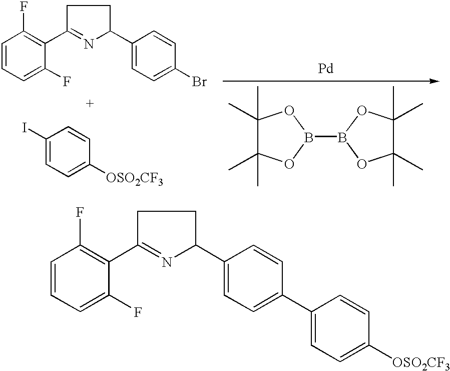

Explanation of the Processes and Intermediates

Process (A)

The formula (II) provides a general definition of the aminoketones required as starting materials for carrying out the process (A) according to the invention. In this formula, R¹, R², R³, R⁴, R⁵, n, r and s preferably, particularly preferably and very particularly preferably have those meanings which have already been mentioned in connection with the description of the compounds of the formula (I) according to the invention as being preferred, particularly preferred, etc., for these radicals.

The aminoketones of the formula (II) are novel. They can be prepared by

a) reacting N-Boc-lactams of the formula (VII)

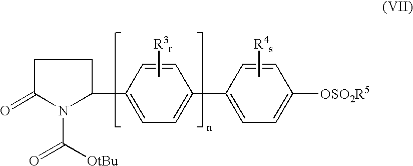

in which

R³, R⁴, R⁵, n, r and s are as defined above

with metallated aromatic compounds of the formula (VIII)

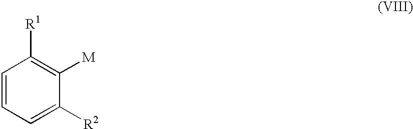

in which

R¹ and R² are as defined above and

M represents Li, MgCl, MgBr, MgI or ZnCl,

if appropriate in the presence of a diluent (for example tetrahydrofuran) at temperatures between −70° C. and +70° C.

The formula (VII) provides a general definition of the N-Boc-lactams required as starting materials for carrying out the process (a). In this formula, R³, R⁴, R⁵, n, r and s preferably, particularly preferably and very particularly preferably have those meanings which have already been mentioned in connection with the description of the compounds of the formula (I) according to the invention as being preferred, particularly preferred, etc., for these radicals.

N-Boc-Lactams of the formula (VII) are novel. They can be prepared, for example, by

b) reacting lactams of the formula (IX)

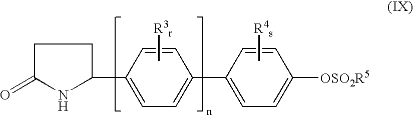

in which

R³, R⁴, R⁵, n, r and s are as defined above

with di-tert-butyl dicarbonate in the presence of a base (for example dimethyl-aminopyridine), if appropriate in the presence of a diluent (for example dichloromethane).

Formula (VIII) provides a general definition of the metallated aromatic compounds required as starting materials for carrying out the process (a). In this formula, R¹ and R² preferably, particularly preferably and very particularly preferably have those meanings which have already been mentioned in connection with the description of the compounds of the formula (I) according to the invention as being preferred, particularly preferred, etc., for these radicals. M preferably represents Li, MgCl, MgBr, MgI, ZnCl, particularly preferably Li, MgCl, MgBr, MgI, very particularly preferably Li, MgCl, MgBr.

Some of the metallated aromatic compounds of the formula (VIII) are known, or they can be prepared by known methods, such as, for example, lithiation or Grignard reaction, from the corresponding aromatic compounds or halogenated aromatic compounds.

The formula (IX) provides a general definition of the lactams required as starting materials for carrying out the process (b). In this formula, R³, R⁴, R⁵, n, r and s preferably, particularly preferably and very particularly preferably have those meanings which have already been mentioned in connection with the description of the compounds of the formula (I) according to the invention as being preferred, particularly preferred, etc., for these radicals.

Lactams of the formula (IX) are novel.

Lactams of the formula (IX-a)

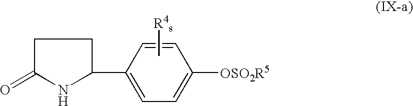

in which

R⁴, R⁵, r and s are as defined above

can be prepared, for example, by

c) reacting lactams of the formula (X)

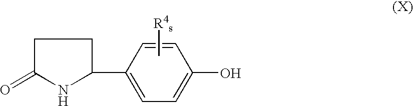

in which

R⁴ and s are as defined above

α) with sulphonyl halides of the formula (XI)

R⁵SO₂—Y (XI)

in which

R⁵ is as defined above and

Y represents halogen, or

β) with sulphonic acid anhydrides of the formula (XII)

(R⁵SO₂)₂O (XII)

in which

R⁵ is as defined above

in the presence of a base and in the presence of a diluent (cf. WO 98/22438, Synthesis 1993, 735 and the literature cited therein).

Lactams of the formula (IX-b)

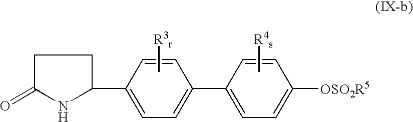

in which

R³, R⁴, R⁵, n, r and s are as defined above

can be prepared, for example, by

d) reacting 5-ethoxy-2-pyrrolidinone with biphenyls of the formula (XIII)

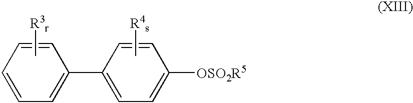

in which

R³, R⁴, R⁵, r and s are as defined above

in the presence of hydrogen fluoride and, if appropriate, in the presence of a diluent (for example dichloromethane).

The formula (X) provides a general definition of the lactams required as starting materials for carrying out the process (c). In this formula, R⁴ and s preferably, particularly preferably and very particularly preferably have those meanings which have already been mentioned in connection with the description of the compounds of the formula (I) according to the invention as being preferred, particularly preferred, etc., for these radicals.

Lactams of the formula (X) are known and/or can be prepared by known process (cf. WO 98/22438).

The formula (XI) provides a general definition of the sulphonyl halides required as starting materials for carrying out the process (c). In this formula, R⁵ preferably, particularly preferably and very particularly preferably has those meanings which have already been mentioned in connection with the description of the compounds of the formula (I) according to the invention as being preferred, particularly preferred, etc., for these radicals. Y preferably represents fluorine or chlorine.

Sulphonyl halides of the formula (XI) are known.

The formula (XII) provides a general definition of the sulphonic acid anhydrides required as starting materials for carrying out the process (c). In this formula, R⁵ preferably, particularly preferably and very particularly preferably has those meanings which have already been mentioned in connection with the description of the compounds of the formula (I) according to the invention as being preferred, particularly preferred, etc., for these radicals.

Sulphonic acid anhydrides of the formula (XII) are known.

The formula (XIII) provides a general definition of the biphenyls required as starting materials for carrying out the process (d). In this formula, R³, R⁴, R⁵, r and s preferably, particularly preferably and very particularly preferably have those meanings which have already been mentioned in connection with the description of the compounds of the formula (I) according to the invention as being preferred, particularly preferred, etc., for these radicals.

Biphenyls of the formula (XIII) are known. They can be prepared, for example, by

e) reacting hydroxybiphenyls of the formula (XIV)

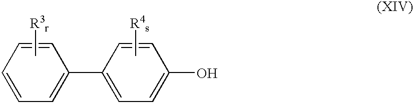

in which

R³, R⁴, r and s are as defined above

e-1) with sulphonyl halides of the formula (XI)

R⁵SO₂—Y (XI)

in which

R⁵ is as defined above and

Y represents halogen or

e-2) with sulphonic acid anhydrides of the formula (XII)

(R⁵SO₂)₂O (XII)

in which

R⁵ is as defined above

in the presence of a base (for example potassium carbonate) and, if appropriate, in the presence of a diluent (for example acetonitrile) (cf. Synthesis 1993, 735 and literature cited therein; see also WO 98/22438).

The formula (XIV) provides a general definition of the hydroxybiphenyls required as starting materials for carrying out the process (e). In this formula, R³, R⁴, r and s preferably, particularly preferably and very particularly preferably have those meanings which have already been mentioned in connection with the description of the compounds of the formula (I) according to the invention as being preferred, particularly preferred, etc., for these radicals.

Hydroxybiphenyls of the formula (XIV) are known.

Suitable diluents for carrying out the process (A) according to the invention are in each case all customary inert organic solvents. Preference is given to using optionally halogenated aliphatic, alicyclic or aromatic hydrocarbons, such as petroleum ether, hexane, heptane, cyclohexane, methylcyclohexane, benzene, toluene, xylene or decalin; chlorobenzene, dichlorobenzene, dichloromethane, chloroform, carbon tetrachloride, dichloroethane or trichloroethane; ethers, such as diethyl ether, diisopropyl ether, methyl t-butyl ether, methyl t-amyl ether, dioxane, tetrahydrofuran, 1,2-dimethoxyethane, 1,2-diethoxyethane or anisole; nitriles, such as acetonitrile, propionitrile, n- or i-butyronitrile or benzonitrile; amides, such as N,N-dimethylformamide, N,N-dimethylacetamide, N-methylformanilide, N-methylpyrrolidone or hexamethylphosphoric triamide; esters, such as methyl acetate or ethyl acetate; sulphoxides, such as dimethyl sulphoxide; or sulphones, such as sulpholane. Particular preference is given to using methylene chloride, chloroform, toluene, methanol or ethanol.

Suitable for carrying out the process (A) according to the invention are in each case all customary Lewis acids or protic acids. Methods for removing Boc are generally known (cf., for example, T. W. Greene, P. G. M. Wuts, Protective Groups in Organic Synthesis, Ed. 3, New York, Wiley & Sons, 1999, pp. 520-525). Preference is given to using trifluoroacetic acid, HCl or HBr for removing the Boc protective group.

When carrying out the process (A) according to the invention, the reaction temperatures can in each case be varied within a relatively wide range. In general, the process is carried out at temperatures between −20° C. and +120° C., preferably between −10° C. and 60° C.

When carrying out the process (A) according to the invention, in general 100 mol of a protic acid are employed per mole of the compound of the formula (II). However, it is also possible to employ the reaction components in other ratios. Work-up is carried out by customary methods. In general, the reaction mixture is concentrated, taken up in a suitable solvent and adjusted to pH 12 using sodium hydroxide, and the organic phase is washed with water, dried over sodium sulphate, filtered and concentrated. If appropriate, the residue is freed from any impurities that may still be present using customary methods, such as chromatography or recrystallization.

To prepare chiral compounds of the formulae (I-c) and (I-d), the lactams of the formula (IX) can be subjected to an optical resolution. This is carried out, for example, using methods of preparative chromatography, preferably the method of High Performance Liquid Chromatography (HPLC). For this purpose, use is made, for example, of a chiral stationary silica gel phase. A silica gel derivative (for example mercaptopropyl silica gel) modified with N-methacryloyl-L-leucine-D-menthylamide has been found to be particularly suitable for separating the compounds of the formula (IX). This separating material is known (cf. EP-A 0 379 917). Suitable mobile phases are all customary inert organic solvents, and mixtures of these. Preference is given to using optionally halogenated aliphatic, alicyclic or aromatic hydrocarbons, such as petroleum ether, hexane, heptane, cyclohexane; dichloromethane, chloroform; alcohols, such as methanol, ethanol, propanol; nitriles, such as acetonitrile; esters, such as methyl acetate or ethyl acetate. Particular preference is given to using aliphatic hydrocarbons, such as hexane or heptane, and alcohols, such as methanol or propanol, very particularly preferably n-heptane and isopropanol, or mixtures of these. The resulting (R)-configured enantiomers are then used as starting materials for the synthesis route described above.

Process (B)

The formula (III) provides a general definition of the (bi)phenols required as starting materials for carrying out the process (B) according to the invention. In this formula, R¹, R², R³, R⁴, n, r and s preferably, particularly preferably and very particularly preferably have those meanings which have already been mentioned in connection with the description of the compounds of the formula (I) according to the invention as being preferred, particularly preferred, etc., for these radicals.

The (bi)phenols of the formula (III) are known and/or can be prepared by known processes (cf. WO 98/22438).

Suitable sulphonylating agents for carrying out the process (B) according to the invention are sulphonyl halides of the formula (XI) and sulphonic acid anhydrides of the formula (XII).

Suitable diluents for carrying out the process (B) according to the invention are in each case all customary inert organic solvents. Preference is given to using optionally halogenated aliphatic, alicyclic or (hetero)aromatic hydrocarbons, such as petroleum ether, hexane, heptane, cyclohexane, methylcyclohexane, benzene, toluene, xylene, pyridine or decalin; chlorobenzene, dichlorobenzene, dichloromethane, chloroform, carbon tetrachloride, dichloroethane or trichloroethane; ethers, such as diethyl ether, diisopropyl ether, methyl t-butyl ether, methyl t-amyl ether, dioxane, tetrahydrofuran, 1,2-dimethoxyethane, 1,2-diethoxyethane or anisole; nitriles, such as acetonitrile, propionitrile, n- or i-butyronitrile or benzonitrile; amides, such as N,N-dimethylformamide, N,N-dimethylacetamide, N-methylformanilide, N-methylpyrrolidone or hexamethylphosphoric triamide; esters, such as methyl acetate or ethyl acetate; sulphoxides, such as dimethyl sulphoxide; or sulphones, such as sulpholane. Particular preference is given to using pyridine, dichloromethane, chloroform, tetrahydrofuran or toluene. If appropriate, it is possible to use mixtures with water and to carry out the reaction in a two-phase system.

Suitable acid binders for carrying out the process (B) according to the invention are all inorganic and organic bases customary for such reactions. Preference is given to using alkaline earth metal or alkali metal hydroxides, such as sodium hydroxide, calcium hydroxide, potassium hydroxide, or else ammonium hydroxide, alkali metal carbonates, such as sodium carbonate, potassium carbonate, potassium bicarbonate, sodium bicarbonate, alkali metal or alkaline earth metal acetates, such as sodium acetate, potassium acetate, calcium acetate and also tertiary amines, such as trimethylamine, triethylamine, tributylamine, diethylisopropylamine, N,N-dimethyl-aniline, pyridine, N-methylpiperidine, N,N-dimethylaminopyridine, diazabicyclooctane (DABCO), diazabicyclononene (DBN) or diazabicycloundecene (DBU). Particular preference is given to using triethylamine, diethylisopropylamine, sodium hydroxide, potassium hydroxide or potassium carbonate.

When carrying out the process (B) according to the invention, the reaction temperatures can in each case be varied within a relatively wide range. In general, the process is carried out at temperatures between −70° C. and +110° C., preferably between −20° C. and +40° C.

When carrying out the process (B) according to the invention, in general 1 mol or a slight excess of sulphonylating agent and from 0.5 to 3 mol of acid binder are employed per mole of the compound of the formula (III). However, it is also possible to use the reaction components in other ratios. Work-up is carried out by customary methods. In general, the reaction mixture is decanted, washed, dried, filtered and concentrated. If appropriate, the residue is freed from any impurities that may still be present using customary methods, such as chromatography or recrystallization.

Process (C)

Formula (IV) provides a general definition of the pyrrolines required as starting materials for carrying out process (C) according to the invention. In this formula, R¹, R², R³ and r preferably, particularly preferably and very particularly preferably have those meanings which have already been mentioned in connection with the description of the compounds of the formula (I) according to the invention as being preferred, particularly preferred, etc., for these radicals.

The pyrrolines of the formula (IV-a)

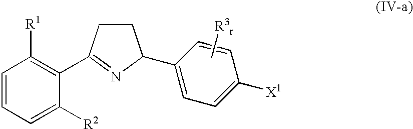

in which

R¹, R², R³ and r are as defined above and

X¹ represents chlorine, bromine or iodine

are known and can be prepared by known processes (cf. WO 98/22438).

The pyrrolines of the formula (IV-b)

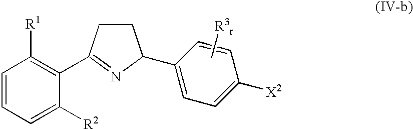

in which

R¹, R², R³ and r are as defined above and

X² represents —OSO₂CF₃ or —OSO₂(CF₂)₃CF₃

are novel.

Pyrrolines of the formula (IV-b) are prepared by process (A).

The formula (VI) provides a general definition of the iodides required as starting materials for carrying out the process (C) according to the invention. In this formula, R⁴, R⁵ and s preferably, particularly preferably and very particularly preferably have those meanings which have already been mentioned in connection with the description of the compounds of the formula (I) according to the invention as being preferred, particularly preferred, etc., for these radicals.

Iodides of the formula (VI) are generally known or commercially available.

For carrying out the process (C) according to the invention, use is generally made of a palladium catalyst which for its part may be used with or without addition of further ligands. The catalyst used is preferably PdCl₂(dppf) [dppf=1,1′-bis(diphenylphosphino)ferrocene], Pd(PPh₃)₄, PdCl₂(PPh₃)₂, PdCl₂(CH₃CN)₂, Pd₂(dba)₃ [dba=dibenzylideneacetone] or Pd(OAc)₂, particularly preferably PdCl₂(dppf), Pd(PPh₃)₄, PdCl₂(PPh₃)₂ or Pd(OAc)₂, very particularly preferably PdCl₂(dppf) or Pd(PPh₃)₄.

Suitable ligands are triarylphosphines, trialkylphosphines or arsines. Preference is given to using dppf, PPh₃, P(t-Bu)₃, Pcy₃ or AsPh₃, particularly preferably dppf.

Suitable diboronic esters are 4,4,4′,4′,5,5,5′,5′-octamethyl-2,2′-bis-1,3,2-dioxaborolane, 5,5,5′,5′-tetramethyl-2,2′-bis-1,3,2-dioxaborinane, 4,4,4′,4′,6,6′-hexamethyl-2,2′-bis-1,3,2-dioxaborinane or 2,2′-bis-1,3,2-benzodioxaborole. All diboronic esters are known.

Acid binders suitable for carrying out the process (C) according to the invention are in each case all inorganic and organic bases customary for such reactions. Preference is given to using alkaline earth metal or alkali metal hydroxides, such as sodium hydroxide, calcium hydroxide, potassium hydroxide, or else ammonium hydroxide, alkali metal carbonates, such as sodium carbonate, potassium carbonate, potassium bicarbonate, sodium bicarbonate, alkali metal or alkaline earth metal acetates, such as sodium acetate, potassium acetate, calcium acetate, alkali metal fluorides, and also tertiary amines, such as trimethylamine, triethylamine, tributylamine, N,N-dimethyl-aniline, pyridine, N-methylpiperidine, N,N-dimethylaminopyridine, diazabicyclooctane (DABCO), diazabicyclononene (DBN) or diazabicycloundecene (DBU). However, it is also possible to carry out the process without additional acid binder, or to employ an excess of the amine component so that it simultaneously acts as acid binder. Particular preference is given to using barium hydroxide, tripotassium phosphate, caesium carbonate, potassium carbonate, sodium carbonate, potassium acetate, triethylamine, potassium tert-butoxide, caesium fluoride or potassium fluoride.

Suitable diluents for carrying out the process (C) according to the invention are in each case all customary inert organic solvents. Preference is given to using optionally halogenated aliphatic, alicyclic or aromatic hydrocarbons, such as petroleum ether, hexane, heptane, cyclohexane, methylcyclohexane, benzene, toluene, xylene or decalin; chlorobenzene, dichlorobenzene, dichloromethane, chloroform, carbon tetrachloride, dichloroethane or trichloroethane; ethers, such as diethyl ether, diisopropyl ether, methyl t-butyl ether, methyl t-amyl ether, dioxane, tetrahydrofuran, 1,2-dimethoxyethane, 1,2-diethoxyethane or anisole; nitriles, such as acetonitrile, propionitrile, n- or i-butyronitrile or benzonitrile; amides, such as N,N-dimethylformamide, N,N-dimethylacetamide, N-methylformanilide, N-methylpyrrolidone or hexamethylphosphoric triamide; esters, such as methyl acetate or ethyl acetate; sulphoxides, such as dimethyl sulphoxide; or sulphones, such as sulpholane. Particular preference is given to using acetone, dimethoxyethane, dioxane, tetrahydrofuran, dimethylformamide, dimethylacetamide, dimethyl sulphoxide, ethanol, toluene or, if appropriate, mixtures of the diluents mentioned with water.

When carrying out the process (C) according to the invention, the reaction temperatures can in each case be varied within a relatively wide range. In general, the process is carried out at temperatures between 0° C. and 140° C., preferably between 10° C. and 120° C., particularly preferably between 20° C. and 120° C.

The process (C) according to the invention can be carried out in two variants. In the first variant, a compound of the formula (IV) is initially reacted with a diboronic ester. This gives intermediates of the formula (V) which are then reacted in a second step with iodides of the formula (VI). In the second variant, the intermediates of the formula (V) are not isolated but reacted in situ with iodides of the formula (VI). In both variants, it is also possible to employ initially the iodides of the formula (VI) and only in the second step the pyrrolines of the formula (IV).

When carrying out the process (C) according to the invention, in general 1 mol or a slight excess of a diboronic ester and 1 mol or a slight excess of iodide of the formula (VI) and 3% of a palladium catalyst are employed per mole of the compound of the formula (IV). However, it is also possible to employ the reaction components in other ratios. The compound of the formula (IV) or, alternatively, the compound of the formula (VI) may be initially charged. Work-up is carried out by customary methods. In general, the reaction mixture is diluted with water and extracted with ethyl acetate. The organic phase is washed with water, dried over sodium sulphate, filtered and concentrated. If appropriate, the residue is freed from any impurities that may still be present using customary methods, such as chromatography or recrystallization. To isolate the intermediates of the formula (V), work-up as described is carried out prior to the addition of the next reaction component.

All processes according to the invention are generally carried out under atmospheric pressure. However, in each case it is also possible to operate under elevated or reduced pressure.

The active compounds of the invention, having good plant tolerance and favourable warm-blood toxicity, are suitable for controlling animal pests, in particular insects, arachnids and nematodes, which are encountered in agriculture, in forests, in the protection of stored products and of materials, and in the hygiene sector. They may be preferably used as crop protection agents. They are active against normally sensitive and resistant species and against all or some stages of development. The abovementioned pests include:

From the order of the Isopoda, for example, Oniscus asellus, Armadillidium vulgare and Porcellio scaber.

From the order of the Diplopoda, for example, Blaniulus guttulatus.

From the order of the Chilopoda, for example, Geophilus carpophagus and Scutigera spp.

From the order of the Symphyla, for example, Scutigerella immaculata.

From the order of the Thysanura, for example, Lepisma saccharina.

From the order of the Collembola, for example, Onychiurus armatus.

From the order of the Orthoptera, for example, Acheta domesticus, Gryllotalpa spp., Locusta migratoria migratorioides, Melanoplus spp. and Schistocerca gregaria.

From the order of the Blattaria, for example, Blatta orientalis, Periplaneta americana, Leucophaea maderae and Blattella germanica.

From the order of the Dermaptera, for example, Forficula auricularia.

From the order of the Isoptera, for example, Reticulitermes spp.

From the order of the Phthiraptera, for example, Pediculus humanus corporis, Haematopinus spp., Linognathus spp., Trichodectes spp. and Damalinia spp.

From the order of the Thysanoptera, for example, Hercinothrips femoralis, Thrips tabaci, Thrips palmi and Frankliniella occidentalis.

From the order of the Heteroptera, for example, Eurygaster spp., Dysdercus intermedius, Piesma quadrata, Cimex lectularius, Rhodnius prolixus and Triatoma spp.

From the order of the Homoptera, for example, Aleurodes brassicae, Bemisia tabaci, Trialeurodes vaporariorum, Aphis gossypii, Brevicoryne brassicae, Cryptomyzus ribis, Aphis fabae, Aphis pomi, Eriosoma lanigerum, Hyalopterus arundinis, Phylloxera vastatrix, Pemphigus spp., Macrosiphum avenae, Myzus spp., Phorodon humuli, Rhopalosiphum padi, Empoasca spp., Euscelis bilobatus, Nephotettix cincticeps, Lecanium comi, Saissetia oleae, Laodelphax striatellus, Nilaparvata lugens, Aonidiella aurantii, Aspidiotus hederae, Pseudococcus spp. and Psylla spp.

From the order of the Lepidoptera, for example, Pectinophora gossypiella, Bupalus piniarius, Cheimatobia brumata, Lithocolletis blancardella, Hyponomeuta padella, Plutella xylostella, Malacosoma neustria, Euproctis chrysorrhoea, Lymantria spp., Bucculatrix thurberiella, Phyllocnistis citrella, Agrotis spp., Euxoa spp., Feltia spp., Earias insulana, Heliothis spp., Mamestra brassicae, Panolis flammea, Spodoptera spp., Trichoplusia ni, Carpocapsa pomonella, Pieris spp., Chilo spp., Pyrausta nubilalis, Ephestia kuehniella, Galleria mellonella, Tineola bisselliella, Tinea pellionella, Hofmannophila pseudospretella, Cacoecia podana, Capua reticulana, Choristoneura fumiferana, Clysia ambiguella, Homona magnanima, Tortrix viridana, Cnaphalocerus spp. and Oulema oryzae.

From the order of the Coleoptera, for example, Anobium punctatum, Rhizopertha dominica, Bruchidius obtectus, Acanthoscelides obtectus, Hylotrupes bajulus, Agelastica alni, Leptinotarsa decemlineata, Phaedon cochleariae, Diabrotica spp., Psylliodes chrysocephala, Epilachna varivestis, Atomaria spp., Oryzaephilus surinamensis, Anthonomus spp., Sitophilus spp., Otiorrhynchus sulcatus, Cosmopolites sordidus, Ceuthorrhynchus assimilis, Hypera postica, Dermestes spp., Trogoderma spp., Anthrenus spp., Attagenus spp., Lyctus spp., Meligethes aeneus, Ptinus spp., Niptus hololeucus, Gibbium psylloides, Tribolium spp., Tenebrio molitor, Agriotes spp., Conoderus spp., Melolontha melolontha, Amphimallon solstitialis, Costelytra zealandica and Lissorhoptrus oryzophilus.

From the order of the Hymenoptera, for example, Diprion spp., Hoplocampa spp., Lasius spp., Monomorium pharaonis and Vespa spp.

From the order of the Diptera, for example, Aedes spp., Anopheles spp., Culex spp., Drosophila melanogaster, Musca spp., Fannia spp., Calliphora erythrocephala, Lucilia spp., Chrysomyia spp., Cuterebra spp., Gastrophilus spp., Hyppobosca spp., Stomoxys spp., Oestrus spp., Hypoderma spp., Tabanus spp., Tannia spp., Bibio hortulanus, Oscinella frit, Phorbia spp., Pegomyia hyoscyami, Ceratitis capitata, Dacus oleae, Tipula paludosa, Hylemyia spp. and Liriomyza spp.

From the order of the Siphonaptera, for example, Xenopsylla cheopis and Ceratophyllus spp.

From the class of the Arachnida, for example, Scorpio maurus, Latrodectus mactans, Acarus siro, Argas spp., Omithodoros spp., Dermanyssus gallinae, Eriophyes ribis, Phyllocoptruta oleivora, Boophilus spp., Rhipicephalus spp., Amblyomma spp., Hyalomma spp., Ixodes spp., Psoroptes spp., Chorioptes spp., Sarcoptes spp., Tarsonemus spp., Bryobia praetiosa, Panonychus spp., Tetranychus spp., Hemitarsonemus spp. and Brevipalpus spp.

The plant-parasitic nematodes include, for example, Pratylenchus spp., Radopholus similis, Ditylenchus dipsaci, Tylenchulus semipenetrans, Heterodera spp., Globodera spp., Meloidogyne spp., Aphelenchoides spp., Longidorus spp., Xiphinema spp., Trichodorus spp. and Bursaphelenchus spp.

The compounds of the formula (I) according to the invention show in particular excellent activity against caterpillars, beetle larvae, spider mites, aphids and leaf miner flies.

At certain concentrations or application rates, the compounds according to the invention may, if appropriate, also be used as herbicides and microbicides, for example as fungicides, antimycotics and bactericides. If appropriate, they may also be used as intermediates or precursors for the synthesis of further active compounds.

All plants and plant parts can be treated in accordance with the invention. Plants are to be understood as meaning in the present context all plants and plant populations such as desired and undesired wild plants or crop plants (inclusive of naturally occurring crop plants). Crop plants can be plants which can be obtained by conventional plant breeding and optimization methods or by biotechnological and genetic engineering methods or by combinations of these methods, inclusive of the transgenic plants and inclusive of the plant cultivars protectable or not protectable by plant breeders' rights. Plant parts are to be understood to mean all above-ground and underground parts and organs of plants, such as shoot, leaf, flower and root, examples which may be mentioned being leaves, needles, stalks, stems, flowers, fruit bodies, fruits, seeds, roots, tubers and rhizomes. The plant parts also include harvested material, and vegetative and generative propagation material, for example cuttings, tubers, rhizomes, offsets and seeds.

Treatment according to the invention of the plants and plant parts with the active compounds is carried out directly or by allowing the compounds to act on their surroundings, environment or storage space by the customary treatment methods, for example by immersion, spraying, evaporation, fogging, scattering, painting on and, in the case of propagation material, in particular in the case of seeds, also by applying one or more coats.

The active compounds according to the invention can be converted into the customary formulations, such as solutions, emulsions, wettable powders, suspensions, powders, dusts, pastes, soluble powders, granules, suspension-emulsion concentrates, natural and synthetic materials impregnated with active compound, and microencapsulations in polymeric substances.

These formulations are produced in a known manner, for example by mixing the active compounds according to the invention with extenders, that is liquid solvents and/or solid carriers, optionally with the use of surfactants, that is emulsifiers and/or dispersants, and/or foam-formers.

If the extender used is water, it is also possible to employ for example organic solvents as auxiliary solvents. Essentially, suitable liquid solvents are: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example petroleum fractions, mineral and vegetable oils, alcohols such as butanol or glycol and also their ethers and esters, ketones such as acetone, methyl ethyl ketone, methyl isobutyl ketone or cyclohexanone, strongly polar solvents such as dimethylformamide and dimethyl sulphoxide, and also water.

Suitable solid carriers are:

for example ammonium salts and ground natural minerals such as kaolins, clays, talc, chalk, quartz, attapulgite, montmorillonite or diatomaceous earth, and ground synthetic minerals, such as finely divided silica, alumina and silicates; suitable solid carriers for granules are: for example crushed and fractionated natural rocks such as calcite, marble, pumice, sepiolite and dolomite, and also synthetic granules of inorganic and organic meals, and granules of organic material such as sawdust, coconut shells, maize cobs and tobacco stalks;

suitable emulsifiers and/or foam-formers are: for example nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates and also protein hydrolysates;

suitable dispersants are: for example lignosulphite waste liquors and methylcellulose.

Tackifiers such as carboxymethylcellulose and natural and synthetic polymers in the form of powders, granules or latices, such as gum arabic, polyvinyl alcohol and polyvinyl acetate, as well as natural phospholipids such as cephalins and lecithins, and synthetic phospholipids, can be used in the formulations. Other additives can be mineral and vegetable oils.

It is possible to use colorants such as inorganic pigments, for example iron oxide, titanium oxide and Prussian Blue, and organic dyes, such as alizarin dyes, azo dyes and metal phthalocyanine dyes, and trace nutrients such as salts of iron, manganese, boron, copper, cobalt, molybdenum and zinc.

The formulations generally comprise between 0.1 and 95% by weight of active compound, preferably between 0.5 and 90%.

The active compounds according to the invention can be used as such or in their formulations as a mixture with other active compounds, such as insecticides, attractants, sterilizing agents, bactericides, acaricides, nematicides, fungicides, growth-regulating substances or herbicides, including known active compounds, to widen the activity spectrum or to prevent the development of resistance, for example. In many cases, synergistic effects are obtained, i.e. the activity of the mixture is greater than the activity of the individual components. The insecticides include, for example, phosphoric acid esters, carbamates, carboxylates, chlorinated hydrocarbons, phenylureas and substances produced by microorganisms, inter alia.

Suitable mixing partners are, for example, the following compounds:

Fungicides:

aldimorph, ampropylfos, ampropylfos-potassium, andoprim, anilazine, azaconazole, azoxystrobin,

benalaxyl, benodanil, benomyl, benzamacril, benzamacryl-isobutyl, bialaphos, binapacryl, biphenyl, bitertanol, blasticidin-S, bromuconazole, bupirimate, buthiobate,

calcium polysulphide, capsimycin, captafol, captan, carbendazim, carboxin, carvon, quinomethionate, chlobenthiazone, chlorfenazole, chloroneb, chloropicrin, chlorothalonil, chlozolinate, clozylacon, cufraneb, cymoxanil, cyproconazole, cyprodinil, cyprofuram,

debacarb, dichlorophen, diclobutrazole, diclofluanid, diclomezine, dicloran, diethofencarb, difenoconazole, dimethirimol, dimethomorph, diniconazole, diniconazole-M, dinocap, diphenylamine, dipyrithione, ditalimfos, dithianon, dodemorph, dodine, drazoxolon,

ediphenphos, epoxiconazole, etaconazole, ethirimol, etridiazole,

famoxadon, fenapanil, fenarimol, fenbuconazole, fenfuram, fenitropan, fenpiclonil, fenpropidin, fenpropimorph, fentin acetate, fentin hydroxide, ferbam, ferimzone, fluazinam, flumetover, fluoromide, fluquinconazole, flurprimidol, flusilazole, flusulfamide, flutolanil, flutriafol, folpet, fosetyl-aluminium, fosetyl-sodium, fthalide, fuberidazole, furalaxyl, furametpyr, furcarbonil, furconazole, furconazole-cis, furmecyclox,

guazatine,

hexachlorobenzene, hexaconazole, hymexazole,

imazalil, imibenconazole, iminoctadine, iminoctadine albesilate, iminoctadine triacetate, iodocarb, ipconazole, iprobenfos (IBP), iprodione, irumamycin, isoprothiolane, isovaledione,

kasugamycin, kresoxim-methyl, copper preparations, such as: copper hydroxide, copper naphthenate, copper oxychloride, copper sulphate, copper oxide, oxine-copper and Bordeaux mixture,

mancopper, mancozeb, maneb, meferimzone, mepanipyrim, mepronil, metalaxyl, metconazole, methasulfocarb, methfuroxam, metiram, metomeclam, metsulfovax, mildiomycin, myclobutanil, myclozolin,

nickel dimethyldithiocarbamate, nitrothal-isopropyl, nuarimol,

ofurace, oxadixyl, oxamocarb, oxolinic acid, oxycarboxim, oxyfenthiin,

paclobutrazole, pefurazoate, penconazole, pencycuron, phosdiphen, picoxystrobin, pimaricin, piperalin, polyoxin, polyoxorim, probenazole, prochloraz, procymidone, propamocarb, propanosine-sodium, propiconazole, propineb, pyraclostrobin, pyrazophos, pyrifenox, pyrimethanil, pyroquilon, pyroxyfur,

quinconazole, quintozene (PCNB),

sulphur and sulphur preparations,

tebuconazole, tecloftalam, tecnazene, tetcyclacis, tetraconazole, thiabendazole, thicyofen, thifluzamides, thiophanate-methyl, thiram, tioxymid, tolclofos-methyl, tolylfluanid, triadimefon, triadimenol, triazbutil, triazoxide, trichlamide, tricyclazole, tridemorph, trifloxystrobin, triflumizole, triforine, triticonazole,

uniconazole,

validamycin A, vinclozolin, viniconazole,

zarilamide, zineb, ziram and also

Dagger G, OK-8705, OK-8801,

α-(1,1-dimethylethyl)-β-(2-phenoxyethyl)-1H-1,2,4-triazole-1-ethanol,

α-(2,4-dichlorophenyl)-β-fluoro-β-propyl-1H-1,2,4-triazole-1-ethanol,

α-(2,4-dichlorophenyl)-β-methoxy-α-methyl-1H-1,2,4-triazole-1-ethanol,

α-(5-methyl-1,3-dioxan-5-yl)-β-[[4-(trifluoromethyl)-phenyl]-methylene]-1H-1,2,4-triazole-1-ethanol,

(5RS,6RS)-6-hydroxy-2,2,7,7-tetramethyl-5-(1H-1,2,4-triazol-1-yl)-3-octanone,

(E)-α-(methoxyimino)-N-methyl-2-phenoxy-phenylacetamide, isopropyl {2-methyl-1-[[[1-(4-methylphenyl)ethyl]amino]carbonyl]propyl}-carbamate,

1-(2,4-dichlorophenyl)-2-(1H-1,2,4-triazol-1-yl)-ethanone O-(phenylmethyl) oxime,

1-(2-methyl-1-naphthalenyl)-1H-pyrrole-2,5-dione,

1-(3,5-dichlorophenyl)-3-(2-propenyl)-2,5-pyrrolidinedione,

1-[(diiodomethyl)-sulphonyl]-4-methyl-benzene,

1-[[2-(2,4-dichlorophenyl)-1,3-dioxolan-2-yl]-methyl]-1H-imidazole,

1-[[2-(4-chlorophenyl)-3-phenyloxiranyl]-methyl]-1H-1,2,4-triazole,

1-[1-[2-[(2,4-dichlorophenyl)-methoxy]-phenyl]-ethenyl]-1H-imidazole,

1-methyl-5-nonyl-2-(phenylmethyl)-3-pyrrolidinol,

2′,6′-dibromo-2-methyl-4′-trifluoromethoxy-4′-trifluoro-methyl-1,3-thiazole-5-carboxanilide,

2,2-dichloro-N-[1-(4-chlorophenyl)ethyl]-1-ethyl-3-methylcyclopropanecarboxamide,

2,6-dichloro-5-(methylthio)-4-pyrimidinyl thiocyanate,

2,6-dichloro-N-(4-trifluoromethylbenzyl)-benzamide,

2,6-dichloro-N-[[4-(trifluoromethyl)-phenyl]-methyl]-benzamide,

2-(2,3,3-triiodo-2-propenyl)-2H-tetrazole,

2-[(1-methylethyl)-sulphonyl]-5-(trichloromethyl)-1,3,4-thiadiazole,

2-[[6-deoxy-4-O-(4-O-methyl-β-D-glycopyranosyl)-α-D-glucopyranosyl]-amino]-4-methoxy-1H-pyrrolo[2,3-d]pyrimidine-5-carbonitrile,

2-aminobutane,

2-bromo-2-(bromomethyl)-pentanedinitrile,

2-chloro-N-(2,3-dihydro-1,1,3-trimethyl-1H-inden-4-yl)-3-pyridinecarboxamide,

2-chloro-N-(2,6-dimethylphenyl)-N-(isothiocyanatomethyl)-acetamide,

2-phenylphenol (OPP),

3,4-dichloro-1-[4-(difluoromethoxy)-phenyl]-1H-pyrrole-2,5-dione,

3,5-dichloro-N-[cyano-[(1-methyl-2-propynyl)-oxy]-methyl]-benzamide,

3-(1,1-dimethylpropyl)-1-oxo-1H-indene-2-carbonitrile,

3-[2-(4-chlorophenyl)-5-ethoxy-3-isoxazolidinyl]-pyridine,

4-chloro-2-cyano-N,N-dimethyl-5-(4-methylphenyl)-1H-imidazole-1-sulphonamide,

4-methyl-tetrazolo[1,5-a]quinazolin-5(4H)-one,

8-(1,1-dimethylethyl)-N-ethyl-N-propyl-1,4-dioxaspiro[4,5]decane-2-methanamine,

8-hydroxyquinoline sulphate,

9H-xanthene-2-[(phenylamino)-carbonyl]-9-carboxylic hydrazide,

bis-(1-methylethyl)-3-methyl-4-[(3-methylbenzoyl)-oxy]-2,5-thiophenedicarboxylate,

cis-1-(4-chlorophenyl)-2-(1H-1,2,4-triazol-1-yl)-cycloheptanol,

cis-4-[3-[4-(1,1-dimethylpropyl)-phenyl-2-methylpropyl]-2,6-dimethyl-morpholine hydrochloride,

ethyl [(4-chlorophenyl)-azo]-cyanoacetate,

potassium hydrogen carbonate,

methanetetrathiol sodium salt,

methyl 1-(2,3-dihydro-2,2-dimethyl-1H-inden-1-yl)-1H-imidazole-5-carboxylate,

methyl N-(2,6-dimethylphenyl)-N-(5-isoxazolylcarbonyl)-DL-alaninate,

methyl N-(chloroacetyl)-N-(2,6-dimethylphenyl)-DL-alaninate,

N-(2,3-dichloro-4-hydroxyphenyl)-1-methylcyclohexanecarboxamide,

N-(2,6-dimethylphenyl)-2-methoxy-N-(tetrahydro-2-oxo-3-furanyl)-acetamide,

N-(2,6-dimethylphenyl)-2-methoxy-N-(tetrahydro-2-oxo-3-thienyl)-acetamide,

N-(2-chloro-4-nitrophenyl)-4-methyl-3-nitro-benzenesulphonamide,

N-(4-cyclohexylphenyl)-1,4,5,6-tetrahydro-2-pyrimidinamine,

N-(4-hexylphenyl)-1,4,5,6-tetrahydro-2-pyrimidinamine,

N-(5-chloro-2-methylphenyl)-2-methoxy-N-(2-oxo-3-oxazolidinyl)-acetamide,

N-(6-methoxy-3-pyridinyl)-cyclopropanecarboxamide,

N-[2,2,2-trichloro-1-[(chloroacetyl)-amino]-ethyl]-benzamide,

N-[3-chloro-4,5-bis-(2-propynyloxy)-phenyl]-N′-methoxy-methanimidamide,

N-formyl-N-hydroxy-DL-alanine sodium salt,

O,O-diethyl [2-(dipropylamino)-2-oxoethyl]-ethylphosphoramidothioate,

O-methyl S-phenyl phenylpropylphosphoramidothioate,

S-methyl 1,2,3-benzothiadiazole-7-carbothioate,

spiro[2H]-1-benzopyran-2,1′(3′H)-isobenzofuran-3′-one,

4-[3,4-dimethoxyphenyl)-3-(4-fluorophenyl)acryloyl]morpholine

Bactericides:

bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulphate and other copper preparations.

Insecticides/Acaricides/Nematicides:

abamectin, acephate, acelamiprid, acrinathrin, alanycarb, aldicarb, aldoxycarb, alphacypermethrin, alphamethrin, amitraz, avermectin, AZ 60541, azadirachtin, azamethiphos, azinphos A, azinphos M, azocyclotin,

Bacillus popilliae, Bacillus sphaericus, Bacillus subtilis, Bacillus thuringiensis, Baculoviruses, Beauveria bassiana, Beauveria tenella, bendiocarb, benfuracarb, bensultap, benzoximate, betacyfluthrin, bifenazate, bifenthrin, bioethanomethrin, biopermethrin, bistrifluron, BPMC, bromophos A, bufencarb, buprofezin, butathiofos, butocarboxim, butylpyridaben,

cadusafos, carbaryl, carbofuran, carbophenothion, carbosulfan, cartap, chloethocarb, chlorethoxyfos, chlorfenapyr, chlorfenvinphos, chlorfluazuron, chlormephos, chlorpyrifos, chlorpyrifos M, chlovaporthrin, chromafenozide, cis-resmethrin, cispermethrin, clocythrin, cloethocarb, clofentezine, clothianidine, cyanophos, cycloprene, cycloprothrin, cyfluthrin, cyhalothrin, cyhexatin, cypermethrin, cyromazine,

deltamethrin, demeton M, demeton S, demeton-S-methyl, diafenthiuron, diazinon, dichlorvos, dicofol, diflubenzuron, dimethoate, dimethylvinphos, diofenolan, disulfoton, docusat-sodium, dofenapyn,

eflusilanate, emamectin, empenthrin, endosulfan, Entomopfthora spp., esfenvalerate, ethiofencarb, ethion, ethoprophos, etofenprox, etoxazole, etrimfos,

fenamiphos, fenazaquin, fenbutatin oxide, fenitrothion, fenothiocarb, fenoxacrim, fenoxycarb, fenpropathrin, fenpyrad, fenpyrithrin, fenpyroximate, fenvalerate, fipronil, fluazinam, fluazuron, flubrocythrinate, flucycloxuron, flucythrinate, flufenoxuron, flumethrin, flutenzine, fluvalinate, fonophos, fosmethilan, fosthiazate, fubfenprox, furathiocarb,

granulosis viruses,

halofenozide, HCH, heptenophos, hexaflumuron, hexythiazox, hydroprene,

imidacloprid, indoxacarb, isazofos, isofenphos, isoxathion, ivermectin,

nuclear polyhedrosis viruses,

lambda-cyhalothrin, lufenuron,

malathion, mecarbam, metaldehyde, methamidophos, Metharhizium anisopliae, Metharhizium flavoviride, methidathion, methiocarb, methoprene, methomyl, methoxyfenozide, metolcarb, metoxadiazone, mevinphos, milbemectin, milbemycin, monocrotophos,

naled, nitenpyram, nithiazine, novaluron,

omethoate, oxamyl, oxydemethon M,

Paecilomyces fumosoroseus, parathion A, parathion M, penmethrin, phenthoate, phorate, phosalone, phosmet, phosphamidon, phoxim, pirimicarb, pirimiphos A, pirimiphos M, profenofos, promecarb, propargite, propoxur, prothiofos, prothoate, pymetrozine, pyraclofos, pyresmethrin, pyrethrum, pyridaben, pyridathion, pyrimidifen, pyriproxyfen,

quinalphos,

ribavirin,

salithion, sebufos, silafluofen, spinosad, spirodiclofen, sulfotep, sulprofos,

tau-fluvalinate, tebufenozide, tebufenpyrad, tebupirimiphos, teflubenzuron, tefluthrin, temephos, temivinphos, terbufos, tetrachlorvinphos, tetradifon, thetacypermethrin, thiacloprid, thiamethoxam, thiapronil, thiatriphos, thiocyclam hydrogen oxalate, thiodicarb, thiofanox, thuringiensin, tralocythrin, tralomethrin, triarathene, triazamate, triazophos, triazurone, trichlophenidine, trichlorfon, triflumuron, trimethacarb,

vamidothion, vaniliprole, Verticillium lecanii,

YI 5302,

zeta-cypermethrin, zolaprofos,

(1R-cis)-[5-(phenylmethyl)-3-furanyl]-methyl 3-[(dihydro-2-oxo-3(2H)-furanylidene)-methyl]-2,2-dimethylcyclopropanecarboxylate,

(3-phenoxyphenyl)-methyl 2,2,3,3-tetramethylcyclopropanecarboxylate,

1-[(2-chloro-5-thiazolyl)methyl]tetrahydro-3,5-dimethyl-N-nitro-1,3,5-triazine-2(1H)-imine,

2-(2-chloro-6-fluorophenyl)-4-[4-(1,1-dimethylethyl)phenyl]-4,5-dihydro-oxazole,

2-(acetyloxy)-3-dodecyl-1,4-naphthalenedione,

2-chloro-N-[[[4-(1-phenylethoxy)-phenyl]-amino]-carbonyl]-benzamide,

2-chloro-N-[[[4-(2,2-dichloro-1,1-difluoroethoxy)-phenyl]-amino]-carbonyl]-benzamide,

3-methylphenyl propylcarbamate,

4-[4-(4-ethoxyphenyl)-4-methylpentyl]-1-fluoro-2-phenoxy-benzene,

4-chloro-2-(1,1-dimethylethyl)-5-[[2-(2,6-dimethyl-4-phenoxyphenoxy)ethyl]thio]-3(2H)-pyridazinone,

4-chloro-2-(2-chloro-2-methylpropyl)-5-[(6-iodo-3-pyridinyl)methoxy]-3(2H)-pyridazinone,

4-chloro-5-[(6-chloro-3-pyridinyl)methoxy]-2-(3,4-dichlorophenyl)-3(2H)-pyridazinone,

Bacillus thuringiensis strain EG-2348,

[2-benzoyl-1-(1,1-dimethylethyl)-hydrazinobenzoic acid,

2,2-dimethyl-3-(2,4-dichlorophenyl)-2-oxo-1-oxaspiro[4.5]dec-3-en-4-yl butanoate,

[3-[(6-chloro-3-pyridinyl)methyl]-2-thiazolidinylidene]-cyanamide,

dihydro-2-(nitromethylene)-2H-1,3-thiazine-3(4H)-carboxaldehyde,

ethyl [2-[[1,6-dihydro-6-oxo-1-(phenylmethyl)-4-pyridazinyl]oxy]ethyl]-carbamate,

N-(3,4,4-trifluoro-1-oxo-3-butenyl)-glycine,

N-(4-chlorophenyl)-3-[4-(difluoromethoxy)phenyl]-4,5-dihydro-4-phenyl-1H-pyrazole-1-carboxamide,

N-[(2-chloro-5-thiazolyl)methyl]-N′-methyl-N″-nitro-guanidine,

N-methyl-N′-(1-methyl-2-propenyl)-1,2-hydrazinedicarbothioamide,

N-methyl-N′-2-propenyl-1,2-hydrazinedicarbothioamide,

O,O-diethyl [2-(dipropylamino)-2-oxoethyl]-ethylphosphoramidothioate,

N-cyanomethyl-4-trifluoromethyl-nicotinamide,

3,5-dichloro-1-(3,3-dichloro-2-propenyloxy)-4-[3-(5-trifluoromethylpyridin-2-yloxy)propoxy]-benzene.

A mixture with other known active compounds, such as herbicides, or with fertilizers and growth regulators is also possible.

The active compounds according to the invention can furthermore be present when used as insecticides in their commercially available formulations and in the use forms prepared from these formulations, as a mixture with synergistic agents. Synergistic agents are compounds which increase the action of the active compounds of the invention, without it being necessary for the synergistic agent added to be actively effective itself.

The active compound content of the use forms prepared from the commercially available formulations can vary within wide limits. The active compound concentration of the use forms can be from 0.0000001 to 95% by weight of active compound, preferably between 0.0001 and 1% by weight.

The compounds are employed in a customary manner appropriate for the use forms.

When used against hygiene pests and pests of stored products, the active compound is distinguished by an excellent residual action on wood and clay as well as a good stability to alkali on limed substrates.

As already mentioned above, it is possible to treat all plants and their parts according to the invention. In a preferred embodiment, wild plant species and plant cultivars, or those obtained by conventional biological breeding, such as crossing or protoplast fusion, and parts thereof, are treated. In a further preferred embodiment, transgenic plants and plant cultivars obtained by genetic engineering, if appropriate in combination with conventional methods (Genetically Modified Organisms), and parts thereof are treated. The term “parts” or “parts of plants” or “plant parts” has been explained above.

Particularly preferably, plants of the plant cultivars which are in each case commercially available or in use are treated according to the invention. Plant cultivars are to be understood as meaning plants having certain traits obtained by conventional breeding, by mutagenesis or by recombinant DNA techniques. These can be cultivars, bio- and genotypes.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions which can be used according to the invention, better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products are possible which exceed the effects which were actually to be expected.

The transgenic plants or plant cultivars (i.e. those obtained by genetic engineering) which are preferably to be treated according to the invention include all plants which, in the genetic modification, received genetic material which imparted particularly advantageously useful traits to these plants. Examples of such traits are better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products. Further and particularly emphasized examples of such traits are a better defence of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, cotton, oilseed rape and also fruit plants (with the fruits apples, pears, citrus fruits and grapevines), and particular emphasis is given to maize, soya beans, potatoes, cotton and oilseed rape. Traits that are emphasized in particular are increased defence of the plants against insects by toxins formed in the plants, in particular those formed by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof) in the plants (hereinbelow referred to as “Bt plants”). Traits that are furthermore particularly emphasized are the increased resistance of plants to fungi, bacteria and viruses by systemic acquired resistance (SAR), systemin, phytoalexins, elicitors and resistance genes, and the corresponding proteins and toxins that are expressed. Traits that are furthermore particularly emphasized are the increased tolerance of the plants to certain herbicidally active compounds, for example imidazolinones, sulphonylureas, glyphosate or phosphinotricin (for example the “PAT” gene). The genes which impart the desired traits in question can also be present in combinations with one another in the transgenic plants. Examples of “Bt plants” which may be mentioned are maize varieties, cotton varieties, soya bean varieties and potato varieties which are sold under the trade names YIELD GARD® (for example maize, cotton, soya bean), KnockOut® (for example maize), StarLink® (for example maize), Bollgard® (cotton), Nucotn® (cotton) and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya bean), Liberty Link® (tolerance to phosphinotricin, for example oilseed rape), IMI® (tolerance to imidazolinones) and STS® (tolerance to sulphonylureas, for example maize). Herbicide-resistant plants (plants bred in a conventional manner for herbicide tolerance) which may be mentioned include the varieties sold under the name Clearfield® (for example maize). Of course, these statements also apply to plant cultivars having these or still-to-be-developed genetic traits, which plants will be developed and/or marketed in the future.

The plants listed can be treated according to the invention in a particularly advantageous manner with the compounds of the general formula (I) or the active compound mixtures according to the invention. The preferred ranges stated above for the active compounds or mixtures also apply to the treatment of these plants. Particular emphasis is given to the treatment of plants with the compounds or the mixtures specifically mentioned in the present text.

The active compounds according to the invention act not only against plant, hygiene and stored product pests, but also in the veterinary medicine sector against animal parasites (ectoparasites), such as hard ticks, soft ticks, mange mites, harvest mites, flies (biting and licking), parasitic fly larvae, lice, hair lice, feather lice and fleas. These parasites include:

From the order of the Anoplurida, for example, Haematopinus spp., Linognathus spp., Pediculus spp., Phtirus spp. and Solenopotes spp.

From the order of the Mallophagida and the suborders Amblycerina and Ischnocerina, for example, Trimenopon spp., Menopon spp., Trinoton spp., Bovicola spp., Werneckiella spp., Lepikentron spp., Damalina spp., Trichodectes spp. and Felicola spp.

From the order Diptera and the suborders Nematocerina and Brachycerina, for example, Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp. and Melophagus spp.

From the order of the Siphonapterida, for example, Pulex spp., Ctenocephalides spp., Xenopsylla spp. and Ceratophyllus spp.

From the order of the Heteropterida, for example, Cimex spp., Triatoma spp., Rhodnius spp. and Panstrongylus spp.

From the order of the Blattarida, for example, Blatta orientalis, Periplaneta americana, Blattella germanica and Supella spp.

From the subclass of the acarids (Acarina) and the orders of the Meta- and Mesostigmata, for example, Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp. and Varroa spp.

From the order of the Actinedida (Prostigmata) and Acaridida (Astigmata), for example, Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp. and Laminosioptes spp.

They are, for example, highly active against the developmental stages of ticks such as, for example, Amblyomma hebraeum, and against parasitic flies, such as, for example, against Lucilia cuprina.

The active compounds of the formula (I) according to the invention are also suitable for controlling arthropods which infest agricultural productive livestock, such as, for example, cattle, sheep, goats, horses, pigs, donkeys, camels, buffalo, rabbits, chickens, turkeys, ducks, geese and bees, other pets, such as, for example, dogs, cats, caged birds and aquarium fish, and also so-called test animals, such as, for example, hamsters, guinea pigs, rats and mice. By controlling these arthropods, cases of death and reduction in productivity (for meat, milk, wool, hides, eggs, honey etc.) should be diminished, so that more economic and easier animal husbandry is possible by use of the active compounds according to the invention.

The active compounds according to the invention are used in the veterinary sector in a known manner by enteral administration in the form of, for example, tablets, capsules, potions, drenches, granules, pastes, boluses, the feed-through process and suppositories, by parenteral administration, such as, for example, by injections (intramuscular, subcutaneous, intravenous, intraperitoneal and the like), implants, by nasal administration, by dermal use in the form, for example, of dipping or bathing, spraying, pouring on and spotting on, washing and powdering, and also with the aid of moulded articles containing the active compound, such as collars, ear marks, tail marks, limb bands, halters, marking devices and the like.

When used for cattle, poultry, pets and the like, the active compounds of the formula (I) according to the invention can be used as formulations (for example powders, emulsions, free-flowing compositions), which comprise the active compounds according to the invention in an amount of 1 to 80% by weight, directly or after 100-to 10 000-fold dilution, or they can be used as a chemical bath.

It has furthermore been found that the compounds according to the invention have a strong insecticidal action against insects which destroy industrial materials.

The following insects may be mentioned as examples and as being preferred—but without any limitation:

Beetles, such as

Hylotrupes bajulus, Chlorophorus pilosis, Anobium punctatum, Xestobium rufovillosum, Ptilinus pecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthes rugicollis, Xyleborus spec., Tryptodendron spec., Apate monachus, Bostrychus capucins, Heterobostrychus brunneus, Sinoxylon spec. and Dinoderus minutus.

Hymenopterons, such as

Sirex juvencus, Urocerus gigas, Urocerus gigas taignus and Urocerus augur.

Termites, such as

Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis and Coptotermes formosanus.

Bristletails, such as Lepisma saccharina.

Industrial materials in the present context are to be understood as meaning non-living materials, such as, preferably, plastics, adhesives, sizes, papers and cards, leather, wood and processed wood products and coating compositions.

Wood and processed wood products are materials to be protected, especially preferably, from insect infestation.

Wood and processed wood products which can be protected by the agent according to the invention or mixtures comprising this are to be understood as meaning, for example:

building timber, wooden beams, railway sleepers, bridge components, boat jetties, wooden vehicles, boxes, pallets, containers, telegraph poles, wood panelling, wooden windows and doors, plywood, chipboard, joinery or wooden products which are used quite generally in house-building or in building joinery.

The active compounds according to the invention can be used as such, in the form of concentrates or in generally customary formulations, such as powders, granules, solutions, suspensions, emulsions or pastes.

The formulations mentioned can be prepared in a manner known per se, for example by mixing the active compounds according to the invention with at least one solvent or diluent, emulsifier, dispersing agent and/or binder or fixing agent, a water repellent, if appropriate siccatives and UV stabilizers and if appropriate dyes and pigments, and also other processing auxiliaries.

The insecticidal compositions or concentrates used for the preservation of wood and wood-derived timber products comprise the active compound according to the invention in a concentration of 0.0001 to 95% by weight, in particular 0.001 to 60% by weight.

The amount of the compositions or concentrates employed depends on the nature and occurrence of the insects and on the medium. The optimum amount employed can be determined for the use in each case by series of tests. In general, however, it is sufficient to employ 0.0001 to 20% by weight, preferably 0.001 to 10% by weight, of the active compound, based on the material to be preserved.

Solvents and/or diluents which are used are an organic chemical solvent or solvent mixture and/or an oily or oil-like organic chemical solvent or solvent mixture of low volatility and/or a polar organic chemical solvent or solvent mixture and/or water, and if appropriate an emulsifier and/or wetting agent.

Organic chemical solvents which are preferably used are oily or oil-like solvents having an evaporation number above 35 and a flash point above 30° C., preferably above 45° C. Substances which are used as such oily or oil-like water-insoluble solvents of low volatility are appropriate mineral oils or aromatic fractions thereof, or solvent mixtures containing mineral oils, preferably white spirit, petroleum and/or alkylbenzene.

Mineral oils having a boiling range from 170 to 220° C., white spirit having a boiling range from 170 to 220° C., spindle oil having a boiling range from 250 to 350° C., petroleum and aromatics having a boiling range from 160 to 280° C., turpentine oil and the like, are advantageously employed.

In a preferred embodiment, liquid aliphatic hydrocarbons having a boiling range from 180 to 210° C. or high-boiling mixtures of aromatic and aliphatic hydrocarbons having a boiling range from 180 to 220° C. and/or spindle oil and/or monochloronaphthalene, preferably α-monochloronaphthalene, are used.

The organic oily or oil-like solvents of low volatility which have an evaporation number above 35 and a flash point above 30° C., preferably above 45° C., can be replaced in part by organic chemical solvents of high or medium volatility, provided that the solvent mixture likewise has an evaporation number above 35 and a flash point above 30° C., preferably above 45° C., and that the insecticide/fungicide mixture is soluble or emulsifiable in this solvent mixture.

According to a preferred embodiment, some of the organic chemical solvent or solvent mixture is replaced by an aliphatic polar organic chemical solvent or solvent mixture. Aliphatic organic chemical solvents containing hydroxyl and/or ester and/or ether groups, such as, for example, glycol ethers, esters or the like, are preferably used.

Organic chemical binders which are used in the context of the present invention are the synthetic resins and/or binding drying oils which are known per se, are water-dilutable and/or are soluble or dispersible or emulsifiable in the organic chemical solvents employed, in particular binders consisting of or comprising an acrylate resin, a vinyl resin, for example polyvinyl acetate, polyester resin, polycondensation or polyaddition resin, polyurethane resin, alkyd resin or modified alkyd resin, phenolic resin, hydrocarbon resin, such as indene-coumarone resin, silicone resin, drying vegetable oils and/or drying oils and/or physically drying binders based on a natural and/or synthetic resin.

The synthetic resin used as the binder can be employed in the form of an emulsion, dispersion or solution. Bitumen or bituminous substances can also be used as binders in an amount of up to 10% by weight. Dyestuffs, pigments, water-repelling agents, odour correctants and inhibitors or anticorrosive agents and the like which are known per se can additionally be employed.

It is preferred according to the invention for the composition or concentrate to comprise, as the organic chemical binder, at least one alkyd resin or modified alkyd resin and/or one drying vegetable oil. Alkyd resins having an oil content of more than 45% by weight, preferably 50 to 68% by weight, are preferably used according to the invention.

All or some of the binder mentioned can be replaced by a fixing agent (mixture) or a plasticizer (mixture). These additives are intended to prevent evaporation of the active compounds and crystallization or precipitation. They preferably replace 0.01 to 30% of the binder (based on 100% of the binder employed).

The plasticizers originate from the chemical classes of phthalic acid esters, such as dibutyl, dioctyl or benzyl butyl phthalate, phosphoric acid esters, such as tributyl phosphate, adipic acid esters, such as di-(2-ethylhexyl) adipate, stearates, such as butyl stearate or amyl stearate, oleates, such as butyl oleate, glycerol ethers or higher molecular weight glycol ethers, glycerol esters and p-toluenesulphonic acid esters.

Fixing agents are based chemically on polyvinyl alkyl ethers, such as, for example, polyvinyl methyl ether or ketones, such as benzophenone or ethylenebenzophenone.

Possible solvents or diluents are, in particular, also water, if appropriate as a mixture with one or more of the abovementioned organic chemical solvents or diluents, emulsifiers and dispersing agents.

Particularly effective preservation of wood is achieved by impregnation processes on a large industrial scale, for example vacuum, double vacuum or pressure processes.

The ready-to-use compositions can also comprise other insecticides, if appropriate, and also one or more fungicides, if appropriate.

Possible additional mixing partners are, preferably, the insecticides and fungicides mentioned in WO 94/29 268. The compounds mentioned in this document are an explicit constituent of the present application.

Especially preferred mixing partners which may be mentioned are insecticides, such as chlorpyriphos, phoxim, silafluofen, alphamethrin, cyfluthrin, cypermethrin, deltamethrin, permethrin, imidacloprid, NI-25, flufenoxuron, hexaflumuron, transfluthrin, thiacloprid, methoxyfenozide and triflumuron, and also fungicides, such as epoxiconazole, hexaconazole, azaconazole, propiconazole, tebuconazole, cyproconazole, metconazole, imazalil, dichlorfluanid, tolylfluanid, 3-iodo-2-propynyl butylcarbamate, N-octyl-isothiazolin-3-one and 4,5-dichloro-N-octylisothiazolin-3-one.

The compounds according to the invention can at the same time be employed for protecting objects which come into contact with salt water or brackish water, in particular hulls, screens, nets, buildings, moorings and signalling systems, against fouling.

Fouling by sessile Oligochaeta, such as Serpulidae, and by shells and species from the Ledamorpha group (goose barnacles), such as various Lepas and Scalpellum species, or by species from the Balanomorpha group (acorn barnacles), such as Balanus or Pollicipes species, increases the frictional drag of ships and, as a consequence, leads to a marked increase in operation costs owing to higher energy consumption and additionally frequent residence in the dry dock.

Apart from fouling by algae, for example Ectocarpus sp. and Ceramium sp., fouling by sessile Entomostraka groups, which come under the generic term Cirripedia (cirriped crustaceans), is of particular importance.

Surprisingly, it has now been found that the compounds according to the invention, alone or in combination with other active compounds, have an outstanding antifouling action.

Using the compounds according to the invention, alone or in combination with other active compounds, allows the use of heavy metals such as, for example, in bis-(trialkyltin) sulphides, tri-n-butyltin laurate, tri-n-butyltin chloride, copper(I) oxide, triethyltin chloride, tri-n-butyl-(2-phenyl-4-chlorophenoxy)tin, tributyltin oxide, molybdenum disulphide, antimony oxide, polymeric butyl titanate, phenyl-(bispyridine)-bismuth chloride, tri-n-butyltin fluoride, manganese ethylenebisthio-carbamate, zinc dimethyldithiocarbamate, zinc ethylenebisthiocarbamate, zinc salts and copper salts of 2-pyridinethiol 1-oxide, bisdimethyldithiocarbamoylzinc ethylene-bisthiocarbamate, zinc oxide, copper(I) ethylene-bisdithiocarbamate, copper thiocyanate, copper naphthenate and tributyltin halides to be dispensed with, or the concentration of these compounds to be substantially reduced.

If appropriate, the ready-to-use antifouling paints can additionally comprise other active compounds, preferably algicides, fungicides, herbicides, molluscicides, or other antifouling active compounds.

Preferably suitable components in combination with the antifouling compositions according to the invention are:

algicides such as

2-tert-butylamino-4-cyclopropylamino-6-methylthio-1,3,5-triazine, dichlorophen, diuron, endothal, fentin acetate, isoproturon, methabenzthiazuron, oxyfluorfen, quinoclamine and terbutryn;

fungicides such as

benzo[b]thiophenecarboxylic acid cyclohexylamide S,S-dioxide, dichlofluanid, fluorfolpet, 3-iodo-2-propynyl butylcarbamate, tolylfluanid and azoles such as azaconazole, cyproconazole, epoxiconazole, hexaconazole, metconazole, propiconazole and tebuconazole;

molluscicides such as

fentin acetate, metaldehyde, methiocarb, niclosamid, thiodicarb and trimethacarb; or conventional antifouling active compounds such as 4,5-dichloro-2-octyl-4-isothiazolin-3-one, diiodomethylparatryl sulphone, 2-(N,N-dimethylthiocarbamoylthio)-5-nitrothiazyl, potassium, copper, sodium and zinc salts of 2-pyridinethiol 1-oxide, pyridine-triphenylborane, tetrabutyldistannoxane, 2,3,5,6-tetrachloro-4-(methylsulphonyl)-pyridine, 2,4,5,6-tetrachloroisophthalonitrile, tetramethylthiuram disulphide and 2,4,6-trichlorophenylmaleimide.

The antifouling compositions used comprise the active compounds according to the invention in a concentration of 0.001 to 50% by weight, in particular 0.01 to 20% by weight.

Moreover, the antifouling compositions according to the invention comprise the customary components such as, for example, those described in Ungerer, Chem. Ind. 1985, 37, 730-732 and Williams, Antifouling Marine Coatings, Noyes, Park Ridge, 1973.

Besides the algicidal, fungicidal, molluscicidal active compounds and insecticidal active compounds according to the invention, antifouling paints comprise, in particular, binders.

Examples of recognized binders are polyvinyl chloride in a solvent system, chlorinated rubber in a solvent system, acrylic resins in a solvent system, in particular in an aqueous system, vinyl chloride/vinyl acetate copolymer systems in the form of aqueous dispersions or in the form of organic solvent systems, butadiene/styrene/acrylonitrile rubbers, drying oils such as linseed oil, resin esters or modified hardened resins in combination with tar or bitumens, asphalt and epoxy compounds, small amounts of chlorine rubber, chlorinated polypropylene and vinyl resins.

If appropriate, paints also comprise inorganic pigments, organic pigments or colorants which are preferably insoluble in salt water. Paints may furthermore comprise materials such as rosin to allow controlled release of the active compounds. Furthermore, the paints may comprise plasticizers, modifiers which affect the rheological properties and other conventional constituents. The compounds according to the invention or the abovementioned mixtures may also be incorporated into self-polishing antifouling systems.

The active compounds according to the invention are also suitable for controlling animal pests, in particular insects, arachnids and mites, which are found in enclosed spaces such as, for example, dwellings, factory halls, offices, vehicle cabins and the like. They can be employed in domestic insecticide products for controlling these pests alone or in combination with other active compounds and auxiliaries. They are active against sensitive and resistant species and against all development stages. These pests include:

From the order of the Scorpionidea, for example, Buthus occitanus.

From the order of the Acarina, for example, Argas persicus, Argas reflexus, Bryobia ssp., Dermanyssus gallinae, Glyciphagus domesticus, Ornithodorus moubat, Rhipicephalus sanguineus, Trombicula alfreddugesi, Neutrombicula autumnalis, Dermatophagoides pteronissimus and Dermatophagoides forinae.

From the order of the Araneae, for example, Aviculariidae and Araneidae.

From the order of the Opiliones, for example, Pseudoscorpiones chelifer, Pseudoscorpiones cheiridium and Opiliones phalangium.

From the order of the Isopoda, for example, Oniscus asellus and Porcellio scaber.

From the order of the Diplopoda, for example, Blaniulus guttulatus and Polydesmus spp.

From the order of the Chilopoda, for example, Geophilus spp.

From the order of the Zygentoma, for example, Ctenolepisma spp., Lepisma saccharina and Lepismodes inquilinus.

From the order of the Blattaria, for example, Blatta orientalis, Blattella germanica, Blattella asahinai, Leucophaea maderae, Panchlora spp., Parcoblatta spp., Periplaneta australasiae, Periplaneta americana, Periplaneta brunnea, Periplaneta fuliginosa and Supella longipalpa.

From the order of the Saltatoria, for example, Acheta domesticus.

From the order of the Dermaptera, for example, Forficula auricularia.

From the order of the Isoptera, for example, Kalotermes spp. and Reticulitermes spp.

From the order of the Psocoptera, for example, Lepinatus spp. and Liposcelis spp.

From the order of the Coleoptera, for example, Anthrenus spp., Attagenus spp., Dermestes spp., Latheticus oryzae, Necrobia spp., Ptinus spp., Rhizopertha dominica, Sitophilus granarius, Sitophilus oryzae, Sitophilus zeamais and Stegobium paniceum.

From the order of the Diptera, for example, Aedes aegypti, Aedes albopictus, Aedes taeniorhynchus, Anopheles spp., Calliphora erythrocephala, Chrysozona pluvialis, Culex quinquefasciatus, Culex pipiens, Culex tarsalis, Drosophila spp., Fannia canicularis, Musca domestica, Phlebotomus spp., Sarcophaga carnaria, Simulium spp., Stomoxys calcitrans and Tipula paludosa.

From the order of the Lepidoptera, for example, Achroia grisella, Galleria mellonella, Plodia interpunctella, Tinea cloacella, Tinea pellionella and Tineola bisselliella.

From the order of the Siphonaptera, for example, Ctenocephalides canis, Ctenocephalides felis, Pulex irritans, Tunga penetrans and Xenopsylla cheopis.

From the order of the Hymenoptera, for example, Camponotus herculeanus, Lasius fuliginosus, Lasius niger, Lasius umbratus, Monomorium pharaonis, Paravespula spp. and Tetramorium caespitum.

From the order of the Anoplura, for example, Pediculus humanus capitis, Pediculus humanus corporis and Phthirus pubis.

From the order of the Heteroptera, for example, Cimex hemipterus, Cimex lectularius, Rhodnius prolixus and Triatoma infestans.

They are used in the household insecticides sector alone or in combination with other suitable active compounds such as phosphoric esters, carbamates, pyrethroids, growth regulators or active compounds from other known classes of insecticides.

They are used in aerosols, pressure-free spray products, for example pump and atomizer sprays, automatic fogging systems, foggers, foams, gels, evaporator products with evaporator tablets made of cellulose or polymer, liquid evaporators, gel and membrane evaporators, propeller-driven evaporators, energy-free, or passive, evaporation systems, moth papers, moth bags and moth gels, as granules or dusts, in baits for spreading or in bait stations.

The preparation and the use of the compounds according to the invention is shown in the examples below.

### PREPARATION EXAMPLES

Process (A)

### Example 1

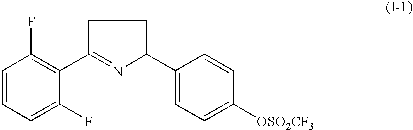

Trifluoroacetic acid (185.37 g, 1.63 mol) is initially charged at 5° C. At this temperature, a solution of 4-[1-[(tert-butoxycarbonyl)amino]-4-(2,6-difluorophenyl)-4-oxobutyl]phenyl trifluoromethanesulphonate (II-1) (42.55 g, 80.8% pure≅0.06 mol) in dichloromethane (150 ml) is added dropwise, and the mixture is then stirred at this temperature for 3 hours. The trifluoroacetic acid is distilled off under reduced pressure and the residue is adjusted to pH 12 using 1N NaOH. The aqueous phase is extracted with dichloromethane (2×100 ml). The combined organic phases are dried over sodium sulphate, filtered and concentrated. The crude product is purified by silica gel chromatography (mobile phase: n-hexane/ethyl acetate 3:1 (v/v)).

This gives 15.83 g (65% of theory) of 4-[5-(2,6-difluorophenyl)-3,4-dihydro-2H-pyrrol-2-yl]phenyl trifluoromethanesulphonate (I-1).

| | |
|---|---|
| HPLC: | log P (pH 2.3) = 3.49 (100% purity). |
| ¹H-NMR: | δ (CD₃CN) = 1.80 (1H, m), 2.65 (1H, m), 3.07 (2H, m), |
| | 5.34 (1H, m), 7.08 (2H, m), 7.37 (2H, d), 7.50 (3H, m) ppm. |

### Example 2

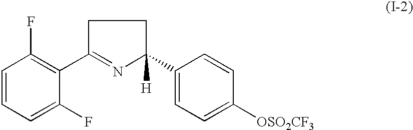

(2R)-4-[5-(2,6-Difluorophenyl)-3,4-dihydro-2H-pyrrol-2-yl]phenyl trifluoromethanesulphonate (I-2) is obtained analogously to Example 1.

| | |
|---|---|
| HPLC: | log P (pH 2.3) = 3.55. |
| Optical rotation: | [α]_{D} = +34.1 (c = 0.92, chloroform), 20° C. |

### Example 3

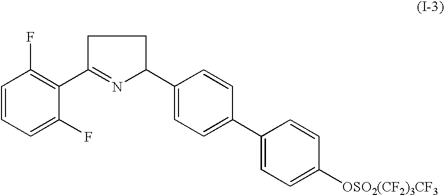

4′-[5-(2,6-Difluorophenyl)-3,4-dihydro-2H-pyrrol-2-yl]-1,1′-biphenyl-4-yl 1,1,2,2,3,3,4,4,4-nonafluoro-1-butanesulphonate (I-3) is obtained analogously to Example 1.

| | |
|---|---|
| HPLC: | log P (pH 2.3) = 5.57 (100% purity). |
| m.p. | 68-69° C. |
| ¹H-NMR: | 67 (CD₃CN) = 1.85 (1H, m), 2.67 (1H, m), 3.06 (2H, m), |
| | 5.34 (1H, m), 7.09 (2H, m), 7.46 (5H, d), 7.65 (2H, d), 7.77 |
| | (2H, d) ppm. |

### Example 4

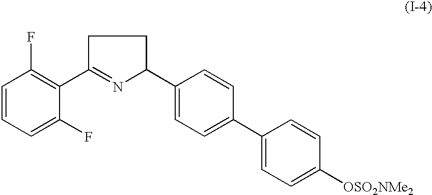

4′-[5-(2,6-Difluorophenyl)-3,4-dihydro-2H-pyrrol-2-yl]-1,1′-biphenyl-4-yl dimethylsulphamate (I-3) is obtained analogously to Example 1. The crude product is purified by silica gel chromatography (mobile phase: cyclohexane/ethyl acetate 1:1) and subsequent trituration with isopropanol.

| | |
|---|---|
| HPLC: | log P (pH 2.3) = 3.06 (100% purity). |
| m.p. | 131-133° C. |
| ¹H-NMR: | δ (CD₃CN) = 1.83 (1H, m), 2.62 (1H, m), 3.06 (2H, m), |
| | 5.33 (1H, m), 7.07 (2H, m), 7.38 (2H, d), 7.42 (2H, d), 7.45 |
| | (1H, m), 7.65 (2H, d), 7.71 (2H, d) ppm. |

Preparation of Starting Materials for Process (A)

Aminoketones of the Formula (II)

### Example (II-1)

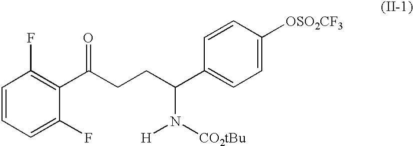

1,3-Difluorobenzene (11.04 g, 96.7 mmol) is initially charged in tetrahydrofuran (100 ml) and cooled to −70° C. At this temperature, n-butyllithium (61.3 ml, 96.7 mmol) is added dropwise. The reaction mixture is warmed to −30° C., and at this temperature, a solution of tert-butyl 2-oxo-5-(4-{[(trifluoromethyl)sulphonyl]oxy}phenyl)-1-pyrrolidinecarboxylate (VI-1) (36.0 g, 87.9 mmol) in tetrahydrofuran (100 ml) is added dropwise. The reaction mixture is stirred at −30° C. for 4 hours and then at room temperature for 16 hours. The reaction mixture is stirred into water (1000 ml) and extracted with ethyl acetate (2×500 ml). The combined organic phases are washed successively with 1N HCl, saturated aqueous sodium bicarbonate solution and saturated aqueous sodium chloride solution. The organic phase is dried over sodium sulphate, filtered and concentrated.

This gives 42.55 g (75% of theory) of 4-[1-[(tert-butoxycarbonyl)amino]-4-(2,6-difluorophenyl)-4-oxobutyl]phenyl trifluoromethanesulphonate (II-1).

| | |
|---|---|
| HPLC: | log P (pH 2.3) = 4.38 (81% purity). |

### Example (II-2)

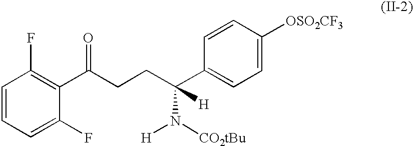

4-[(1R)-1-[(tert-Butoxycarbonyl)amino]-4-(2,6-difluorophenyl)-4-oxobutyl]phenyl trifluoromethanesulphonate (II-2) is obtained analogously to Example (II-1).

| | |
|---|---|
| HPLC: | log P (pH 2.3) = 4.37. |

Beispiel (II-3)

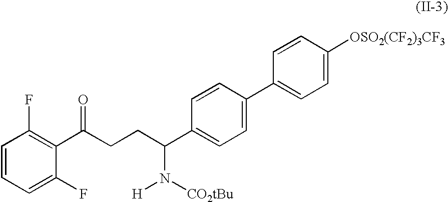

4′-[1-[(tert-Butoxycarbonyl)amino]-4-(2,6-difluorophenyl)-4-oxobutyl]-1,1′-biphenyl-4-yl 1,1,2,2,3,3,4,4,4-nonafluoro-1-butanesulphonate (II-3) is obtained analogously to Example (II-1). The crude product is purified by silica gel chromatography (mobile phase: n-hexane/ethyl acetate 9:1).

| | |
|---|---|
| HPLC: | log P (pH 2.3) = 6.05 (97.60% purity). |
| m.p. | 90-92° C. |

### Example (II-4)

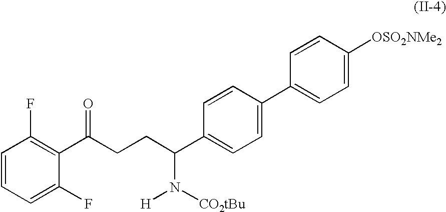

tert-Butyl 4-(2,6-difluorophenyl)-1-(4′-{[(dimethylamino)sulphonyl]oxy}-1,1′-biphenyl-4-yl)-4-oxobutylcarbamate (II-4) is obtained analogously to Example (II-1).

| | |
|---|---|
| HPLC: | log P (pH 2.3) = 4.25. |

N-Boc-lactams of the Formula (VII)

### Example (VII-1)

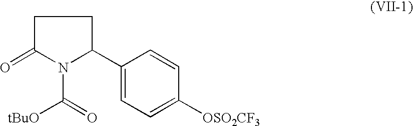

At 0° C., 4-(5-oxo-2-pyrrolidinyl)phenyl trifluoromethanesulphonate (154.63 g, 0.50 mol) is, under an atmosphere of argon, initially charged in dichloromethane (600 ml). At this temperature, di-tert-butyl dicarbonate (218.25 g, 1.0 mol) and dimethylaminopyridine (6.10 g, 0.05 mol) are added successively. The mixture is then stirred at room temperature for 48 hours. The reaction mixture is washed successively with 1N HCl (2×500 ml), saturated aqueous sodium bicarbonate solution and saturated aqueous sodium chloride solution. The organic phase is dried over sodium sulphate, filtered and concentrated.

This gives 198.70 g (99% of theory) of tert-butyl 2-oxo-5-(4-{[(trifluoromethyl)-sulphonyl]oxy}phenyl)-1-pyrrolidinecarboxylate (VII-1).

| | |
|---|---|
| HPLC: | log P (pH 2.3) = 3.31 (98.80% purity). |

### Example (VII-2)

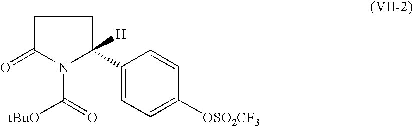

The racemate of 4-(5-oxo-2-pyrrolidinyl)phenyl trifluoromethanesulphonate is separated at room temperature by liquid chromatography on a chiral stationary polyamide silica gel phase (based on the monomer N-methacryloyl-L-leucine-d-menthylamide, particle size: 10 μm, column dimensions: 450 mm×75 mm) using the mobile phase ethyl acetate (flow rate: 100 ml/min) and photometric detection (λ=254 nm). For chromatography, a solution of 40 g of the racemate in 1 l of ethyl acetate is prepared. 6 g (=150 ml of the solution) are chromatographed. Following analytical examination for enantiomeric purity, the appropriate fractions of the eluate are combined and substantially concentrated under reduced pressure and the residue is filtered off and, after washing with n-heptane, dried.

This gives 4-(5-oxo-(2R)-2-pyrrolidinyl)phenyl trifluoromethanesulphonate.

| | |
|---|---|
| Optical rotation: | [α]_{D} = +22.8 (c = 0.9, methanol), 20° C. |

The (R)-enantiomer is then reacted analogously to Example (VII-1), giving tert-butyl 2-oxo-(5R)-5-(4-{[(trifluoromethyl)sulphonyl]oxy}phenyl)-1-pyrrolidinecarboxylate (VII-2).

| | |
|---|---|
| HPLC: | log P (pH 2.3) = 3.31. |

### Example (VII-3)

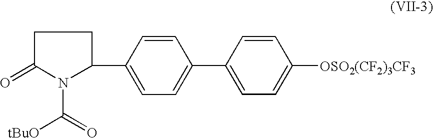

tert-Butyl 2-(4′-{[(1,1,2,2,3,3,4,4,4-nonafluorobutyl)sulphonyl]oxy}-1,1′-biphenyl-4-yl)-5-oxo-1-pyrrolidinecarboxylate (VII-3) is obtained analogously to Example (VII-1). The crude product is purified by silica gel chromatography (mobile phase: n-hexane/ethyl acetate 3:1).

| | |
|---|---|
| HPLC: | log P (pH 2.3) = 5.44 (98.6% purity). |
| m.p. | 115-117° C. |

### Example (VII-4)

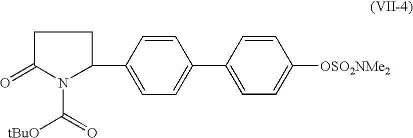

tert-Butyl 2-(4′-{[(dimethylamino)sulphonyl]oxy}-1,1′-biphenyl-4-yl)-5-oxo-1-pyrrolidinecarboxylate (VII-4) is obtained analogously to Example (VII-1). The crude product is recrystallized from isopropanol.

| | |
|---|---|
| HPLC: | log P (pH 2.3) = 3.31 (98.17% purity) |
| m.p. | 171-173° C. |

Lactams of the Formula (IX-b)

### Example (IX-b-1)

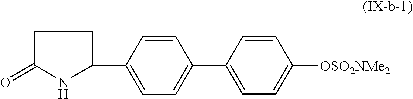

Hydrogen fluoride (50 ml) is initially charged at 0° C. A solution of 5-ethoxy-2-pyrrolidinone (2.58 g, 0.02 mol) and 1,1′-biphenyl-4-yl dimethylsulphamate (XII-2) (2.77 g, 0.01 mol) in dichloromethane (15 ml) is added dropwise, and the reaction mixture is then stirred at room temperature. HF is removed under reduced pressure and the residue is taken up in dichloromethane and washed with saturated aqueous sodium bicarbonate solution. The organic phase is dried over magnesium sulphate, filtered and concentrated. The crude product is reacted further without purification.

This gives 3.20 g (59% of theory) of 4′-(5-oxo-2-pyrrolidinyl)-1,1′-biphenyl-4-yl dimethylsulphamate (IX-b-1) as a mixture with the “ortho” isomer 2′-(5-oxo-2-pyrrolidinyl)-1,1′-biphenyl-4-yl dimethylsulphamate.

| | |
|---|---|
| HPLC (IX-b-1): | log P (pH 2.3) = 2.23 (66.49% purity) |
| HPLC (“ortho” isomer): | log P (pH 2.3) = 2.28 (23.81% purity) |

### Example (IX-b-2)

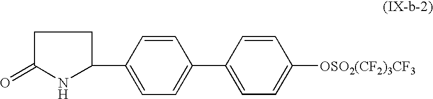

4′-(5-Oxo-2-pyrrolidinyl)-1,1′-biphenyl-4-yl 1,1,2,2,3,3,4,4,4-nonafluoro-1-butanesulphonate (IX-b-2) is obtained analogously to Example (IX-b-1).

| | |
|---|---|
| HPLC: | log P (pH 2.3) = 4.21 (88.60% purity) |

Biphenyls of the Formula (XIII)

### Example (XIII-1)

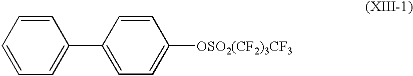

4-Hydroxybiphenyl (1.70 g, 0.01 mol) and potassium carbonate (1.66 g, 0.012 mol) are suspended in acetonitrile (50 ml). 1,1,2,2,3,3,4,4,4-Nonafluorobutane-1-sulphonyl fluoride (3.60 g, 0.01 mol) is added dropwise at room temperature, and the mixture is then stirred at room temperature for 16 hours. The acetonitrile is distilled off, and water is added to the residue. The precipitate is filtered off with suction and dried.

This gives 4.45 g (99% of theory) of 1,1′-biphenyl-4-yl 1,1,2,2,3,3,4,4,4-nonafluoro-1-butanesulphonate (XIII-1).

| | |
|---|---|
| HPLC: | log P(pH 2.3) = 5.81(100% purity) |

### Example (XIII-2)

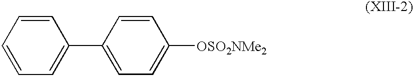

4-Hydroxybiphenyl (1.70 g, 0.01 mol) and potassium carbonate (1.66 g, 0.012 mol) are suspended in acetonitrile (50 ml), stirred at room temperature for 1.5 hours and then cooled to 0° C. Dimethylsulphamoyl chloride (1.44 g, 0.01 mol) is added dropwise, and the reaction mixture is stirred at room temperature for 16 hours. The precipitate is filtered off with suction and the acetonitrile is distilled off. The residue is recrystallized from isopropanol.

This gives 1.81 g (65% of theory) of 1,1′-biphenyl-4-yl dimethylsulphamate (XIII-2) of melting point 105-107° C.

| | |
|---|---|
| HPLC: | log P(pH 2.3) = 3.51(99.6% purity) |

Process (B)

### Example 5

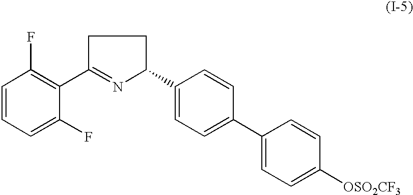

4′-[(2R)-5-(2,6-Difluorophenyl)-3,4-dihydro-2H-pyrrol-2-yl]-1,1′-biphenyl-4-ol (7.0 g, 0.02 mol) is suspended in toluene (150 ml). At room temperature, aqueous sodium hydroxide solution (1.3 ml, 45% strength) is added, and the mixture is stirred at this temperature for another 45 min. Trifluoromethanesulphonyl chloride (2.34 ml, 0.022 mol) is added dropwise, and the mixture is stirred at room temperature for 1.5 h. The toluene phase is decanted off from the solid residue, washed successively with 1 N NaOH, saturated aqueous sodium bicarbonate solution and saturated aqueous ammonium chloride solution, dried over sodium sulphate, filtered and concentrated under reduced pressure. The crude product is purified by silica gel chromatography (mobile phase: cyclohexane/ethyl acetate, 9:1 v/v) and then by preparative HPLC (Kromasil 100-5 C18, 250×50 mm; CH₃CN/H₂O, 80:20 v/v).

This gives 2.36 g (25% of theory) of 4′-[(2R)-5-(2,6-difluorophenyl)-3,4-dihydro-2H-pyrrol-2-yl]-1,1′-biphenyl-4-yl trifluoromethanesulphonate (1-4) of melting point 109° C.

| | |
|---|---|
| HPLC: | log P(pH 2.3) = 4.27(99.88% purity) |
| Optical rotation: | [α]_{D} = +31.8(c = 0.39, MeOH); 20° C. |

### Example 6

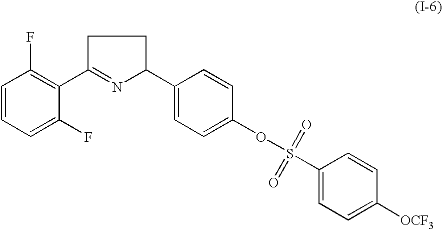

4-[5-(2,6-Difluorophenyl)-3,4-dihydro-2H-pyrrol-2-yl]phenol (2.00 g, 7.3 mmol) is suspended in toluene (30 ml). Aqueous sodium hydroxide solution (0.7 ml, 45% strength, v/v) and 4-(trifluoromethoxy)benzenesulphonyl chloride (1.90 g, 7.3 mmol) are added successively at room temperature. The reaction mixture is stirred at 45° C. for 12 hours. After cooling to room temperature, the phases are separated. The aqueous phase is extracted with ethyl acetate. The combined organic phases are washed with water, dried over sodium sulphate, filtered and concentrated. The crude product is purified by silica gel chromatography (mobile phase: toluene/ethyl acetate, 9:1 v/v).

This gives 2.55 g (70% of theory) of 4-[5-(2,6-difluorophenyl)-3,4-dihydro-2H-pyrrol-2-yl]phenyl 4-(trifluoromethoxy)benzenesulphonate (I-5).

| | |
|---|---|
| HPLC: | log P(pH 2.3) = 3.83(98.6% purity) |
| ¹H-NMR: | δ(CD₃CN) = 1.75(1H, m), 2.60(1H, m), |
| | 3.05(2H, m), 5.26(1H, m), 7.01(2H, d), 7.07(2H, m), |
| | 7.31(2H, d), 7.48(1H, m), 7.50(2H, d), |
| | 7.93(2H, d) ppm. |

Process (C)

### Example 7

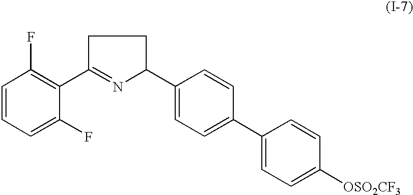

Under an atmosphere of argon, 5-(2,6-difluorophenyl)-2-[4-(4,4,5,5-tetramethyl-1,3,2-dioxaborolan-2-yl)phenyl]-3,4-dihydro-2H-pyrrole (V-I) (0.96 g, 2.5 mmol), 4-iodophenyl trifluoromethanesulphonate (1.06 g, 3.0 mmol), aqueous sodium bicarbonate solution (5 ml, 2 M) and PdCl₂dppf (56 mg, 0.075 mmol) in dimethoxyethane (30 ml) are heated at 60° C. for 1.5 hours and then at 80° C. for 1.5 hours. After cooling, water and ethyl acetate are added to the reaction mixture. The organic phase is dried over sodium sulphate and filtered. Florisil (5 g) is added and the mixture is concentrated to dryness. The crude product is purified by silica gel chromatography (mobile phase: cyclohexane/ethyl acetate, 9:1 v/v). This gives 0.62 g (52% of theory) of 4′-[5-(2,6-difluorophenyl)-3,4-dihydro-2H-pyrrol-2-yl]-1,1′-biphenyl-4-yl trifluoromethanesulphonate (I-7).

| | |
|---|---|
| HPLC: | log P(pH 2.3) = 4.15(96% purity) |
| m.p. | 73-75° C. |
| ¹H-NMR: | δ(CD₃CN) = 1.88(1H, m), 2.68(1H, m), 3.06(2H, m), |
| | 5.35(1H, m), 7.08(2H, d), 7.47(5H, m), |
| | 7.65(2H, m), 7.78(2H, d) ppm. |

Also obtained is a fraction of compound (I-7) of 0.20 g (14% of theory) having a purity of 87%.

Preparation of Starting Materials for Process (C)

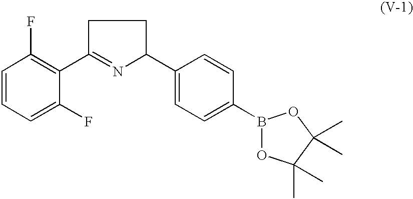

Under an atmosphere of argon, 4-[5-(2,6-difluorophenyl)-3,4-dihydro-2H-pyrrol-2-yl]phenyl trifluoromethanesulphonate (I-1) (60.80 g, 0.15 mol), bispinacolatodiboron (41.90 g, 0.165 mol), potassium acetate (44.20 g, 0.45 mol), PdCl₂dppf (3.30 g, 4.50 mmol) and diphenylphosphinoferrocene (2.50 g, 4.50 mmol) in dioxane (900 ml) are heated at 80° C. for 16 hours. After cooling, the reaction mixture is stirred into water (1000 ml) and extracted with ethyl acetate (2×600 ml). The combined organic phases are dried over sodium sulphate and filtered. Florisil (200 g) is added, and the mixture is evaporated to dryness. The crude product is purified by silica gel chromatography (mobile phase: cyclohexane/ethyl acetate, 4:1 v/v).

This gives 52.80 g (89% of theory) of 5-(2,6-difluorophenyl)-2-[4-(4,4,5,5-tetramethyl-1,3,2-dioxaborolan-2-yl)phenyl]-3,4-dihydro-2H-pyrrole (V-1).

| | |
|---|---|
| HPLC: | log P(pH 7.5) = 4.46(96.7% purity) |
| ¹H-NMR: | δ(CD₃CN) = 1.32(12H, s), 1.78(1H, m), |
| | 2.59(1H, m), 3.03(2H, m), 5.30(1H, m), 7.07(2H, d), |
| | 7.35(2H, m), 7.46(1H, m), 7.70(2H, d) ppm. |

The logP values given in the tables and preparation examples above are determined in accordance with EEC Directive 79/831 Annex V.A8 by HPLC (High Performance Liquid Chromatography) using a reversed-phase column (C 18). Temperature: 43° C.

In the acidic range, the determination is carried out at pH 2.3 using the mobile phases 0.1% aqueous phosphoric acid and acetonitrile; linear gradient from 10% acetonitrile to 90% acetonitrile.

In the neutral range, the determination is carried out at pH 7.5 using the mobile phases 0.01 molar aqueous phosphate buffer solution and acetonitrile; linear gradient from 10% acetonitrile to 90% acetonitrile.

Calibration is carried out using unbranched alkan-2-ones (of 3 to 16 carbon atoms) with known logP values (determination of the logP values by the retention times using linear interpolation between two successive alkanones).

The lambda-max values were determined in the maxima of the chromatographic signals using the UV spectra from 200 nm to 400 nm.

### USE EXAMPLES

### Example A

Aphis gossypii test

| | |
|---|---|
| Solvent: | 30 parts by weight of dimethylformamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Cotton leaves (Gossypium hirsutum) which are heavily infested by the cotton aphid (Aphis gossypii) are treated by being dipped into the preparation of active compound of the desired concentration.

After the desired period of time, the kill in % is determined. 100% means that all aphids have been killed; 0% means that none of the aphids have been killed.

In this test, for example, the following compounds of the Preparation Examples show good activity:

Active compounds, active compound concentrations and test results are shown in the table below.

TABLE A
Aphis gossypii test

| | | Concentration | |
|---|---|---|---|
| | | of active | |
| | | compound | Kill rate in % |
| Active compound | | in ppm | after 6 days |
| (I-1) | 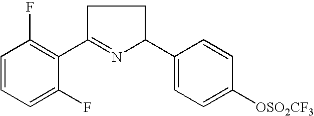 | 200 | 95 |
| (I-2) | 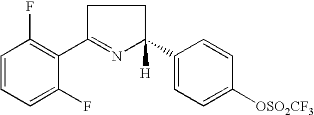 | 100 | 90 |

### Example B

Heliothis virescens test

| | |
|---|---|
| Solvent: | 30 parts by weight of dimethylformamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Soya bean shoots (Glycine max) are treated by being dipped into the preparation of active compound of the desired concentration and are populated with Heliothis virescens caterpillars while the leaves are still moist.

After the desired period of time, the kill in % is determined. 100% means that all caterpillars have been killed; 0% means that none of the caterpillars have been killed.

Active compounds, active compound concentrations and test results are shown in the table below.

TABLE B
Heliothis virescens test

| | | Concentration of | |
|---|---|---|---|
| | | active compound | Kill rate in % |
| Active compound | | in ppm | after 6 days |
| (I-5) | 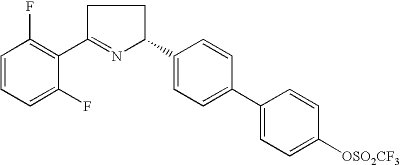 | 5 | 100 |
| (I-7) | 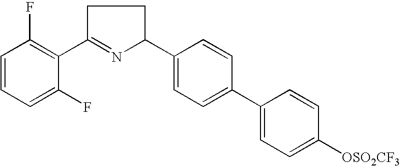 | 1000 | 100 |

### Example C

Myzus test

| | |
|---|---|
| Solvent: | 30 parts by weight of dimethylformamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Cabbage leaves (Brassica oleracea) which are heavily infested by the peach aphid (Myzus persicae) are treated by being dipped into the preparation of active compound of the desired concentration.

After the desired period of time, the kill in % is determined. 100% means that all aphids have been killed; 0% means that none of the aphids have been killed.

Active compounds, active compound concentrations and test results are shown in the table below.

TABLE C
Myzus test

| | | Concentration of | |
|---|---|---|---|
| | | active compound | Kill rate in % |
| Active compound | | in ppm | after 6 days |
| (I-1) | 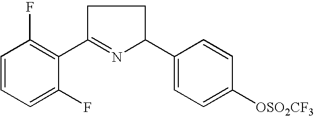 | 20 | 95 |
| (I-2) | 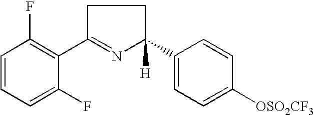 | 500 | 98 |
| (I-6) | 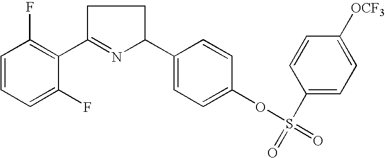 | 1000 | 95 |

### Example D

Meloidogyne test

| | |
|---|---|
| Solvent: | 30 parts by weight of dimethylformamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Vessels are filled with sand, solution of active compound, Meloidogyne incognita egg/larvae suspension and lettuce seeds. The lettuce seeds germinate and the plants develop. On the roots, galls are formed.

After the desired period of time, the nematicidal action is determined in % by the formation of galls. 100% means that no galls were found; 0% means that the number of galls on the treated plants corresponds to that of the untreated control.

Active compounds, active compound concentrations and test results are shown in the table below.

TABLE D
Meloidogyne test

| | | Concentration of | |
|---|---|---|---|
| | | active compound | Kill rate in % |
| Active compound | | in ppm | after 14 days |
| (I-5) | 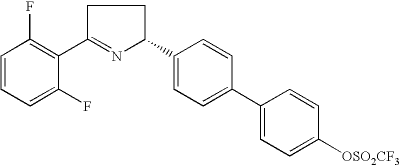 | 20 | 100 |

### Example E

Panonychus test

| | |
|---|---|
| Solvent: | 3 parts by weight of dimethylformamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Plum trees (Prunus domestica) of a height of about 30 cm which are heavily infested by all stages of the fruit tree spider mite (Panonychus ulmi) are sprayed with a preparation of active compound of the desired concentration.

After the desired period of time, the activity in % is determined. 100% means that all spider mites have been killed; 0% means that none of the spider mites have been killed.

Active compounds, active compound concentrations and test results are shown in the table below.

TABLE F
Panonychus test

| | | Concentration of | |
|---|---|---|---|
| | | active compound | Kill rate in % |
| Active compound | | in ppm | after 7 days |
| (I-2) | 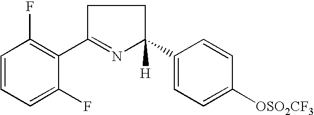 | 200 | 98 |

### Example F

Phaedon larvae test

| | |
|---|---|
| Solvent: | 30 parts by weight of dimethylformamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Cabbage leaves (Brassica oleracea) are treated by being dipped into the preparation of active compound of the desired concentration and are populated with larvae of the mustard beetle (Phaedon cochleariae) while the leaves are still moist.

After the desired period of time, the kill in % is determined. 100% means that all beetle larvae have been killed; 0% means that none of the beetle larvae have been killed.

Active compounds, active compound concentrations and test results are shown in the table below.

TABLE F
Phaedon larvae test

| | | Concentration | |
|---|---|---|---|
| | | of active | |
| | | compound in | Kill rate in % |
| Active compound | | ppm | after 7 days |
| (I-1) | 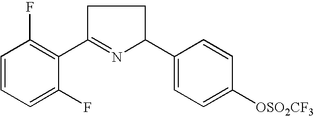 | 1000 | 100 |
| (I-2) | 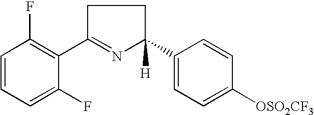 | 1000 | 100 |
| (I-3) | 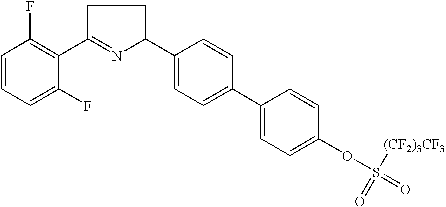 | 1000 | 100 |
| (I-4) | 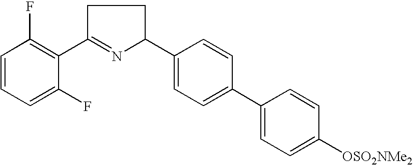 | 1000 | 100 |
| (I-5) | 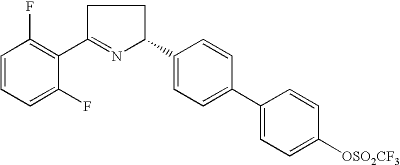 | 1000 | 100 |
| (I-6) | 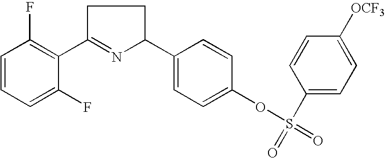 | 1000 | 90 |
| (I-7) | 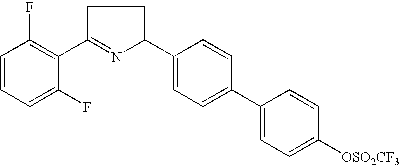 | 1000 | 100 |

### Example G

Plutella test

| | |
|---|---|
| Solvent: | 30 parts by weight of dimethylformamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Cabbage leaves (Brassica oleracea) are treated by being dipped into the preparation of active compound of the desired concentration and are populated with caterpillars of the diamondback moth (Plutella xylostella) while the leaves are still moist.

After the desired period of time, the kill in % is determined. 100% means that all caterpillars have been killed; 0% means that none of the caterpillars have been killed.

Active compounds, active compound concentrations and test results are shown in the table below.

TABLE G
Plutella test

| | | Concentration of | |
|---|---|---|---|
| | | active compound | Kill rate in % |
| Active compound | | in ppm | after 6 days |
| (I-2) | 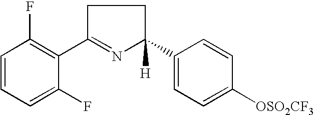 | 500 | 100 |
| (I-5) | 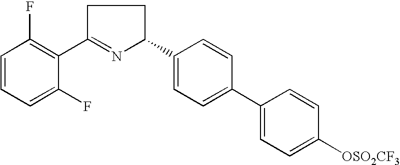 | 5 | 100 |
| (I-7) | 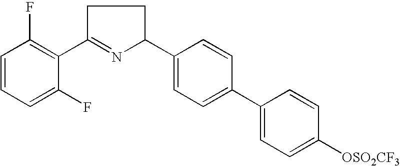 | 1000 | 100 |

### Example H

Spodoptera exigua test

| | |
|---|---|
| Solvent: | 30 parts by weight of dimethylformamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Cabbage leaves (Brassica oleracea) are treated by being dipped into the preparation of active compound of the desired concentration and are populated with larvae of the armyworm (Spodoptera exigua) while the leaves are still moist.

After the desired period of time, the kill in % is determined. 100% means that all larvae have been killed; 0% means that none of the larvae have been killed.

Active compounds, active compound concentrations and test results are shown in the table below.

TABLE H
Spodoptera exigua test

| | | Concentration of | |
|---|---|---|---|
| | | active compound | Kill rate in % |
| Active compound | | in ppm | after 6 days |
| (I-2) | 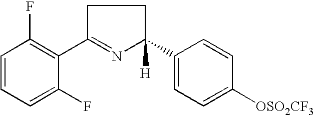 | 500 | 100 |
| (I-7) | 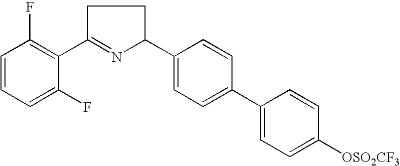 | 1000 | 100 |

### Example I

Spodoptera frugiperda test

| | |
|---|---|
| Solvent: | 30 parts by weight of dimethylformamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Cabbage leaves (Brassica oleracea) are treated by being dipped into the preparation of active compound of the desired concentration and are populated with larvae of the armyworm (Spodoptera frugiperda) while the leaves are still moist.

After the desired period of time, the kill in % is determined. 100% means that all larvae have been killed; 0% means that none of the larvae have been killed.

Active compounds, active compound concentrations and test results are shown in the table below.

TABLE I
Spodoptera frugiperda test

| | | Concentration | |
|---|---|---|---|
| | | of active | |
| | | compound in | Kill rate in % |
| Active compound | | ppm | after 6 days |
| (I-2) | 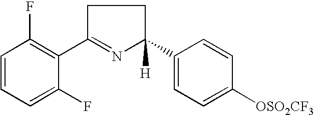 | 500 | 95 |
| (I-3) | 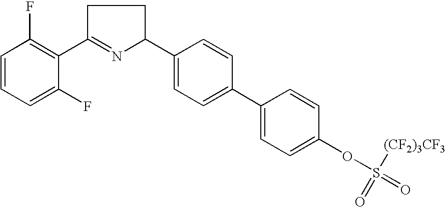 | 1000 | 100 |
| (I-4) | 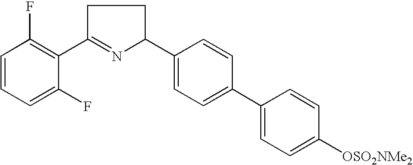 | 1000 | 100 |
| (I-5) | 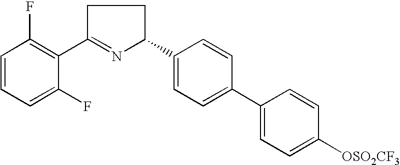 | 1000 | 100 |
| (I-7) | 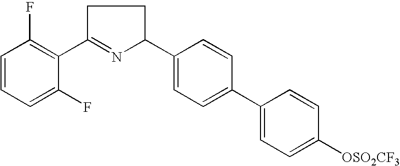 | 1000 | 100 |

### Example J

Tetranychus test (OP-resistant/dip treatment)

| | |
|---|---|
| Solvent: | 30 parts by weight of dimethylformamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Bean plants (Phaseolus vulgaris) which are heavily infested by all stages of the greenhouse red spider mite (Tetranychus urticae) are dipped into a preparation of active compound of the desired concentration.

After the desired period of time, the activity in % is determined. 100% means that all spider mites have been killed; 0% means that none of the spider mites have been killed.

Active compounds, active compound concentrations and test results are shown in the table below.

TABLE J
Tetranychus test

| | | Concentration | |
|---|---|---|---|
| | | of active | |
| | | compound in | Kill rate in % |
| Active compound | | ppm | after 7 days |
| (I-1) | 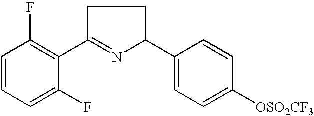 | 100 | 95 |
| (I-2) | 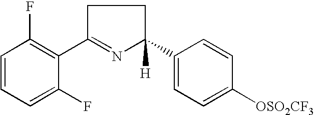 | 100 | 98 |
| (I-3) | 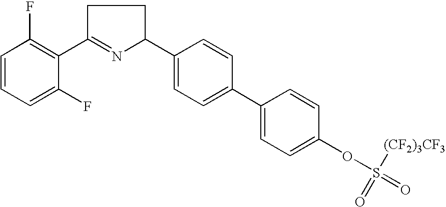 | 100 | 98 |
| (I-6) | 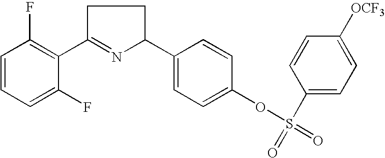 | 100 | 98 |
| (I-7) | 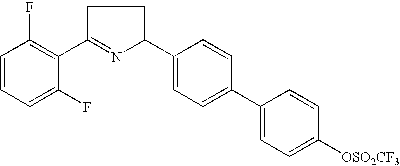 | 100 | 98 |

### Example K

Diabrotica balteata test (larvae in soil)
Critical concentration test/soil insects-
treatment of transgenic plants

| | |
|---|---|
| Solvent: | 7 parts by weight of dimethylformamide |
| Emulsifier: | 1 part by weight of alkylaryl polyglycol ether |

To produce a suitable preparation of active compound, 1 part by weight of active compound is mixed with the stated amount of solvent, the stated amount of emulsifier is added and the concentrate is diluted with water to the desired concentration.

The preparation of active compound is poured onto the soil. Here, the concentration of active compound in the preparation is virtually immaterial, only the amount by weight of active compound per volume unit of soil, which is stated in ppm (mg/l) matters. The soil is filled into 0.25 l pots, and these are allowed to stand at 20° C.

Immediately after the preparation, 5 pregerminated maize corns of the cultivar YIELD GUARD (trademark of Monsanto Comp., USA) are placed into each pot. After 2 days, the appropriate test insects are placed into the treated soil. After a further 7 days, the efficacy of the active compound is determined by counting the maize plants that have emerged (1 plant=20% activity).

### Example L

Heliothis virescens test
(treatment of transgenic plants)

| | |
|---|---|
| Solvent: | 7 parts by weight of dimethylformamide |
| Emulsifier: | 1 part by weight of alkylaryl polyglycol ether |

To produce a suitable preparation of active compound, 1 part by weight of active compound is mixed with the stated amount of solvent and the stated amount of emulsifier, and the concentrate is diluted with water to the desired concentration.

Soya bean shoots (Glycine max) of the cultivar Roundup Ready (trademark of Monsanto Comp., USA) are treated by being dipped into the preparation of active compound of the desired concentration and are populated with the tobacco budworm Heliothis virescens while the leaves are still moist.

After the desired period of time, the kill in % is determined. 100% means that all caterpillars have been killed; 0% means that none of the caterpillars have been killed.

### Example M

Blowfly larvae test/Development-inhibitory action
Lucilia cuprina (48 h/DI)

| | |
|---|---|
| Test animals: | Lucilia cuprina larvae |
| Solvent: | dimethyl sulphoxide |

20 mg of active compound are dissolved in one ml of dimethyl sulphoxide. To prepare a suitable formulation, the solution of active compound is diluted with water to the concentration desired in each case.

About 20 Lucilia cuprina larvae are introduced into a test tube which contains about 1 cm³ of horsemeat and 0.5 ml of the preparation of active compound to be tested. The activity of the preparation of active compound is determined after 48 hours as the mortality of the larvae in %.

The test tubes are then transferred into a beaker whose bottom is covered with sand. After a further 12 days, the test tubes are removed and the pupae and flies are counted. The development-inhibitory action is stated as inhibition of hatching in % (ratio of pupae to flies that have hatched) after 1.5 times the development period of an untreated control.

Active compounds, active compound concentrations and test results are shown in the table below.

TABLE M
Blowfly larvae test

| | | Concentration | |
|---|---|---|---|
| | | of active | |
| | | compound in | Action in % |
| Active compound | | ppm | after 48 hours |
| (I-1) | 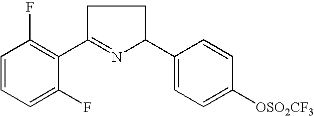 | 100 | 100 |
| (I-2) | 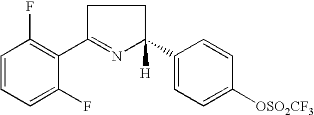 | 100 | 100 |
| (I-5) | 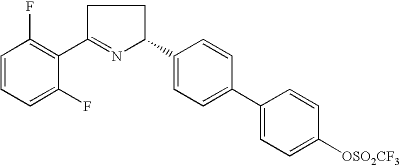 | 100 | 100 |
| (I-6) | 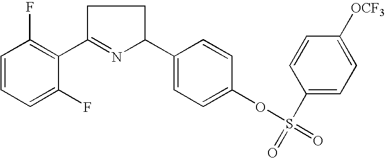 | 100 | 90 |
| (I-7) | 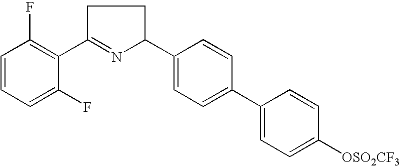 | 100 | 100 |

### Example N

Test with Polyphagous Tick Nymphs, Dip Treatment

Amblyomma hebraeum (DI)

Test with polyphagous tick nymphs, dip treatment
Amblyomma hebraeum (DI)

| | |
|---|---|
| Test animals: | Amblyomma hebraeum nymphs which have |
| | sucked themselves full |
| Solvent: | dimethyl sulphoxide |

20 mg of active compound are dissolved in one ml of dimethyl sulphoxide. To prepare a suitable formulation, the solution of active compound is diluted with water to the concentration desired in each case.

10 nymphs which have sucked themselves full are immersed for 1 minute into the preparation of active compound to be tested. The animals are transferred to Petri dishes (Ø 9.5 cm) fitted with filter discs and covered. After 4 weeks of storage in a climatized room, the mortality is determined.

100% means that none of the animals has undergone normal ecdysis. 0% means that all animals have undergone ecdysis.

In this test, for example, the compounds (I-1), (I-2), (I-5) and (I-7) of the preparation examples show good activity.

1. Δ¹-Pyrrolines of the formula (I) 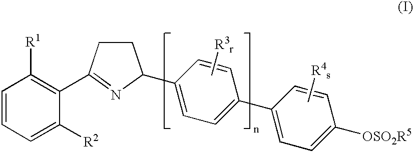
in which
n represents 0 or 1,
r and s independently of one another represent 0, 1 or 2,
R¹ represents halogen or methyl,
R² represents hydrogen or halogen,
R³ and R⁴ independently of one another represent halogen, alkyl, haloalkyl, alkoxy or haloalkoxy,
R⁵ represents alkyl, haloalkyl, phenyl which is in each case optionally mono- or polysubstituted by identical or different radicals from the list W¹ or represents —NR⁶R⁷,
W¹ represents halogen, alkyl, haloalkyl, alkoxy, haloalkoxy, alkyl-carbonyl, alkoxycarbonyl or —S(O)_{q}R⁸,
R⁶ represents alkyl or haloalkyl,
R⁷ represents hydrogen, alkyl or haloalkyl,
R⁶ and R⁷ furthermore together represent alkylene or alkoxyalkylene,
R⁸ represents alkyl or haloalkyl and
q represents 0, 1 or 2.

2. Compounds of formula (I) according to claim 1 in which
n represents 0 or 1,
r and s independently of one another represent 0, 1 or 2,
R¹ represents fluorine, chlorine, bromine or methyl,
R² represents hydrogen, fluorine, chlorine or bromine,
R³ and R⁴ independently of one another represent fluorine, chlorine, bromine, C₁-C₆-alkyl, C₁-C₆-haloalkyl, C₁-C₆-alkoxy or C₁-C₆-haloalkoxy,
R⁵ represents C₁-C₆-alkyl, C₁-C₆-haloalkyl, phenyl which is in each case optionally mono- to tetrasubstituted by identical or different radicals from the list W¹ or represents —NR⁶R⁷,
W¹ represents fluorine, chlorine, bromine, C₁-C₆-alkyl, C₁-C₆-haloalkyl, C₁-C₆-alkoxy, C₁-C₆-haloalkoxy, C₁-C₆-alkylcarbonyl, C₁-C₆-alkoxy-carbonyl or —S(O)_{q}R⁸,
R⁶ represents C₁-C₆-alkyl or C₁-C₆-haloalkyl,
R⁷ represents hydrogen, C₁-C₆-alkyl or C₁-C₆-haloalkyl,
R⁶ and R⁷ furthermore together represent C₃-C₆-alkylene or C₁-C₄-alkoxy-C₁-C₄-alkylene,
R⁸ represents C₁-C₆-alkyl or C₁-C₆-haloalkyl and
q represents 0, 1 or 2.

3. Compounds of the formula (I) according to claim 1 in which
n represents 0 or 1,
r and s independently of one another represent 0, 1 or 2,
R¹ represents fluorine, chlorine or methyl,
R² represents hydrogen, fluorine or chlorine,
R³ and R⁴ independently of one another represent fluorine, chlorine, C₁-C₄-alkyl, C₁-C₄-haloalkyl, C₁-C₄-alkoxy, C₁-C₄-haloalkoxy,
R⁵ represents C₁-C₄-alkyl, C₁-C₄-haloalkyl, phenyl which is in each case optionally mono- to trisubstituted by identical or different radicals from the list W¹ or represents —NR⁶R⁷,
W¹ represents fluorine, chlorine, C₁-C₄-alkyl, C₁-C₄-haloalkyl, C₁-C₄-alkoxy, C₁-C₄-haloalkoxy, C₁-C₄-alkylcarbonyl, C₁-C₄-alkoxycarbonyl or —S(O)_{q}R⁸,
R⁶ represents C₁-C₄-alkyl or C₁-C₄-haloalkyl,
R⁷ represents hydrogen, C₁-C₄-alkyl or C₁-C₄-haloalkyl,
R⁶ and R⁷ furthermore together represent C₄-C₅-alkylene or —(CH₂)₂—O—(CH₂)₂—,
R⁸ represents C₁-C₄-alkyl or C₁-C₄-haloalkyl and
q represents 0, 1 or 2.

4. Compounds of the formula (I) according to claim 1 in which
n represents 0 or 1,
r and s independently of one another represent 0 or 1,
R¹ represents fluorine or chlorine,
R² represents hydrogen or fluorine,
R³ and R⁴ independently of one another represent fluorine, chlorine, methyl, ethyl, n-propyl, i-propyl, n-butyl, i-butyl, s-butyl, t-butyl, trifluoromethyl, trifluoroethyl, methoxy, ethoxy, n-propoxy, i-propoxy, n-butoxy, i-butoxy, s-butoxy, t-butoxy, trifluoromethoxy or trifluoroethoxy,
R⁵ represents methyl, ethyl, n-propyl, i-propyl, n-butyl, i-butyl, s-butyl, t-butyl, trifluoromethyl, trifluoroethyl, nonafluorobutyl, phenyl which is in each case optionally mono- or disubstituted by identical or different radicals from the list W¹ or represents —NR⁶R⁷,
W¹ represents fluorine, chlorine, methyl, ethyl, n-propyl, i-propyl, n-butyl, i-butyl, s-butyl, t-butyl, trifluoromethyl, trifluoroethyl, methoxy, ethoxy, n-propoxy, i-propoxy, n-butoxy, i-butoxy, s-butoxy, t-butoxy, trifluoromethoxy, trifluoroethoxy, —COCH₃, —CO₂CH₃, —SCF₃, —SCHF₂, —SOCF₃, —SOCHF₂, —SO₂CF₃ or —SO₂CHF₂,
R⁶ represents methyl, ethyl, n-propyl, i-propyl, n-butyl, i-butyl, s-butyl, t-butyl, trifluoromethyl or trifluoroethyl and
R⁷ represents hydrogen, methyl, ethyl, n-propyl, i-propyl, n-butyl, i-butyl, s-butyl, t-butyl, trifluoromethyl or trifluoroethyl.

5. Compounds of the formula (I-a) 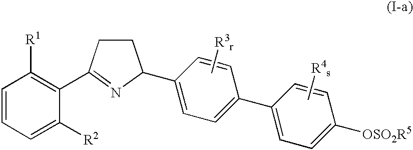
in which
R¹, R², R³, R⁴, R⁵, r and s are as defined in any of claims 1 to 4.

6. Compounds of the formula (I-b) 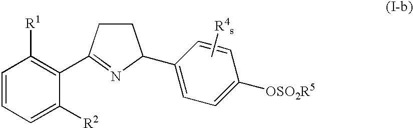
in which
R¹, R², R⁴, R⁵ and s are as defined in any of claims 1 to 4.

7. Δ¹-Pyrrolines of the formula (I-c) 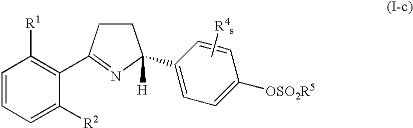
in which
R¹, R², R⁴, R⁵ and s are as defined in any of claims 1 to 4.

8. Δ¹-Pyrrolines of the formula (I-d) 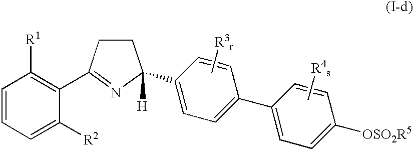
in which
R¹, R², R³, R⁴, R⁵, r and s are as defined in any of claims 1 to 4.

9. Process for preparing compounds of the formula (I) according to claim 1, characterized in that
A) aminoketones of the formula (II) 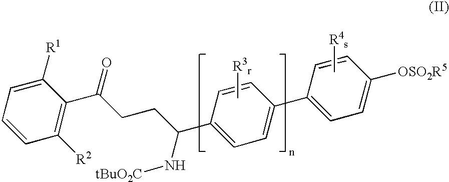
in which R¹, R², R³, R⁴, R⁵, n, r and s are as defined in claim 1
are treated with a Lewis acid or a protic acid, or
B) (bi)phenols of the formula (III) 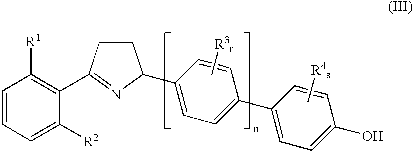
in which
R¹, R², R³, R⁴, n, r and s are as defined in claim 1
are reacted with a sulphonylating agent, if appropriate in the presence of an acid binder and if appropriate in the presence of a diluent, or
C) Δ¹-pyrrolines of the formula (I-a) 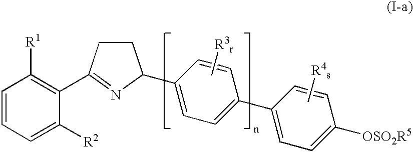
in which
n represents 1 and
R¹, R², R³, R⁴, R⁵, r and s are as defined in claim 1
are obtained by reacting pyrrolines of the formula (IV) 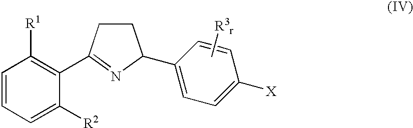
in which
R¹, R², R³ and r are as defined above and
X represents Br, Cl, I, —OSO₂CF₃ or —OSO₂(CF₂)₃CF₃,
initially with a diboronic ester in the presence of a catalyst, in the presence of an acid binder and, if appropriate, in the presence of a diluent and, if appropriate after prior isolation of the resulting compounds of the formula (V) 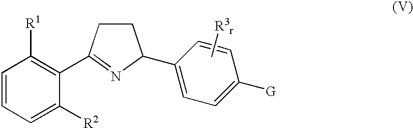
in which
R¹, R², R³ and r are as defined above and
G represents 4,4,5,5-tetramethyl-1,3,2-dioxaborolan-2-yl, 5,5-dimethyl-1,3,2-dioxaborinan-2-yl, 4,4,6-trimethyl-1,3,2-dioxaborinan-2-yl or 1,3,2-benzodioxaborol-2-yl,
reacting with iodides of the formula (VI) 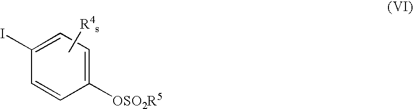
in which
R⁴, R⁵ and s are as defined in claim 1
in the presence of a catalyst, in the presence of a diboronic ester, in the presence of an acid binder and, if appropriate, in the presence of a diluent.

10. Aminoketones of the formula (II) 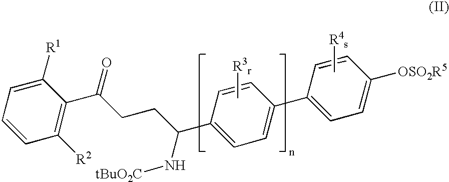
in which R¹, R², R³, R⁴, R⁵, n, r and s are as defined in any of claims 1 to 4.

11. Pyrrolines of the formula (IV-b) 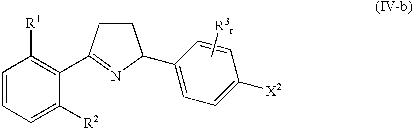
in which
R¹, R², R³ and r are as defined in any of claims 1 to 4 and
X² represents —OSO₂CF₃.

12. Pyrrolines of the formula (IV-b) 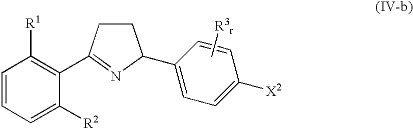
in which
R¹, R², R³ and r are as defined in any of claims 1 to 4 and
X² represents —OSO₂(CF₂)₃CF₃.

13. N-Boc-lactams of the formula (VII) 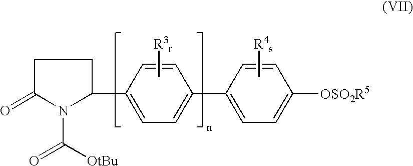
in which
R³, R⁴, R⁵, n, r and s are as defined in any of claims 1 to 4.

14. Lactams of the formula (IX) 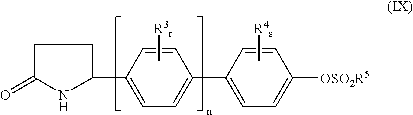
in which
R³, R⁴, R⁵, n, r and s are as defined in any of claims 1 to 4.

15. Pesticides, characterized in that they comprise at least one compound of the formula (I) according to claim 1, in addition to extenders and/or surfactants.

16. Use of compounds of the formula (I) according to claim 1 for controlling pests.

17. Method for controlling pests, characterized in that compounds of the formula (I) according to claim 1 are allowed to act on pests and/or their habitat.

18. Process for preparing pesticides, characterized in that compounds of the formula (I) according to claim 1 are mixed with extenders and/or surfactants.

19. Use of compounds of the formula (I) according to claim 1 for preparing pesticides.
